(19) United States
(12) Patent Application Publication
(10) Pub. No.: US 2026/0176364 A1
(43) Pub. Date: Jun. 25, 2026

(54) GENERATION OF ANTIBODIES ACTING AS SILENT AND POSITIVE ALLOSTERIC MODULATORS OF THE ALPHA7 NICOTINIC ACETYLCHOLINE RECEPTOR

(71) Applicants: INSTITUT PASTEUR, PARIS (FR); CENTRE NATIONAL DE LA RECHERCHE SCIENTIFIQUE, PARIS (FR); LANZHOU INSTITUTE OF BIOLOGICAL PRODUCTS CO., LTD., LANZHOU (CN)

(72) Inventors: Qimeng LI, LANZHOU (CN); Pierre-Jean CORRINGER, PARIS (FR); Akos NEMECZ, TORUN (PL); Gabriel AYME, PARIS (FR); Pierre LAFAYE, PARIS (FR); Marie PREVOST, PARIS (FR); Nathalie BARILONE, PARIS (FR)

(21) Appl. No.: 19/127,174

(22) PCT Filed: Nov. 10, 2023
(86) PCT No.: PCT/IB2023/000654

§ 371 Date: May 5, 2025

Related U.S. Application Data
(60) Provisional application No. 63/383,099, filed on Nov. 10, 2022.

(51) Int. Cl. C07K 16/28; G01N 33/68

(52) U.S. Cl. CPC C07K 16/286; C07K 16/2881; G01N 33/6896; C07K 2317/35; C07K 2317/565; C07K 2317/569; C07K 2317/92; C07K 2319/00; G01N 2333/70571

(57) ABSTRACT

The invention relates to compositions and methods for producing and using antibodies against the alpha7 nicotinic acetylcholine receptor.

### BACKGROUND OF THE INVENTION

Nicotinic acetylcholine receptors (nAChRs) belong to the pentameric ligand-gated ion channel family and play a key role in neuronal communication as well as in non-neuronal cells such as immune and epithelial cells. The major nAChRs in the brain and at the periphery are the homomeric α7-nAChR, and the heteromeric α4β2- and α3β4-nAChRs, a fraction of these later incorporating the accessory α5 or β3 subunits (Nemecz et al., 2016). Acetylcholine (ACh) binding promotes a global reorganization in nAChRs, whereupon their intrinsic channel opens, while the prolonged binding of ACh promotes a second reorganization, where the channel closes in what is termed the desensitized state. Among nAChRs, α7-nAChR displays unique properties, including low probability of channel opening and rapid desensitization (Bouzat et al., 2018).

The α7 nicotinic acetylcholine receptor (nAChR) is a pentameric ligand-gated ion channel mediating communication between neuronal and non-neuronal cells. It is a potential drug target for treating cognitive disorders and inflammation. In this context, small molecules acting as positive allosteric modulators (PAMs) binding outside the acetylcholine site have attracted considerable interest.

The α7-nAChR has attracted considerable interest and been pursued as a potential therapeutic target for numerous indications (Papke and Horenstein, 2021). The α7-nAChR is abundant in brain regions such as the hippocampus and the prefrontal cortex that are important for cognitive functions, therefore drugs that activate or potentiate the receptor have been shown to be effective in preclinical models for cognitive disorders. Additionally, several therapeutics were tested through clinical trials in the context of Alzheimer's and Parkinson's diseases, as well as schizophrenia (Terry and Callahan, 2020). However, as of yet there has not been any approval for clinical use, either due to lack of efficacy or to adverse effects. The α7-nAChR is also an essential component of the cholinergic anti-inflammatory pathway, specifically its activation through excitation of the vagus nerve triggers release of anti-inflammatory cytokines (Wang et al., 2003). Of note, the α7-nAChR is not only found as homopentamers in the brain, but also as heteromers in complex with the β2 subunit (Wu et al., 2016), as well as with the dupα7 subunit, which is a truncated subunit lacking part of the N-terminal extracellular ligand-binding domain and is associated with neurological disorders, including schizophrenia and immunomodulation (Lasala et al., 2018).

A lot of effort has been dedicated to developing small molecules specifically targeting the α7-nAChRs. Each nAChR subunit within the pentamer is composed of an extracellular domain (ECD) folded as a β sandwich, a transmembrane domain (TMD) consisting of four α-helices, and an intracellular domain (ICD) consisting of two helices and a variably sized poorly resolved domain connecting the two (Noviello et al., 2021) (Zhao et al., 2021). The endogenous neurotransmitter's (ACh) binding sites, also called orthosteric sites, are located at all of the subunit interfaces within the ECD of the homomeric α7-nAChR. Agonists, partial agonists, and antagonists all bind at the orthosteric site and were the first therapeutic focus, whereas negative (NAM) and positive (PAM) allosteric modulators binding outside of this site have also actively been investigated more recently (Papke and Horenstein, 2021). Indeed, the very rapid desensitization of α7-nAChRs is expected to strongly limit the efficacy of conventional agonists, while allosteric modulators can potentially overcome this issue. In addition, PAMs and NAMs are expected to better maintain the spatio-temporal characteristics of endogenous ACh activation and to target non-conserved sites, increasing the chemical diversity of active compounds.

In chronological order, calcium was first identified as a PAM (Galzi et al., 1996) (Natarajan et al., 2020) binding in the lower part of the ECD (Le Novère et al., 2002) (Noviello et al., 2021). Ivermectin was then identified as a PAM binding in the TMD (Krause et al., n.d.). Ivermectin can be classified as a type I PAM, potentiating the ACh-elicited current at the peak of the electrophysiological response but not impairing the downstream desensitization process. Subsequently, large series of small molecules binding at the TMD were found to strongly modulate the receptor, as exemplified by the type II PAM PNU-120596, that not only potentiates the ACh-elicited currents but also inhibits desensitization to a large extent (Hurst, 2005) (Zhao et al., 2021). Additionally, the PAM 4BP-TQS can even activate the receptor by itself, thereby having both agonistic and modulatory properties outside of the orthosteric site (Ago-PAM) (Gill et al., 2011). Finally, several modulatory sites for small fragments were identified at different levels of the ECD but have not, as of yet, been exploited for drug-design purpose (Spurny et al., 2015) (Delbart et al., 2018).

In addition to small molecules, an interest has recently grown around single-domain antibody fragments of camelids, generally termed nanobodies, in developing biotechnologies (Jovčevska and Muyldermans, 2020). Nanobodies correspond to the variable domain (VHH) of the heavy chain-only antibodies expressed in these animals. Moreover, they usually bind to surface cavities (Uchański et al., 2020) and motifs that often reorganize during conformational transitions of the receptor, thereby acting as conformation-specific ligands that is the basis of their allosteric modulation. In addition, nanobodies have a number of advantages over small molecules, notably a usually high affinity typically in the nanomolar range, as well as a high specificity conferred by the large surface of nanobody-antigen interaction. As an illustration within the pLGIC family, nanobodies acting as positive and negative allosteric modulators were reported on the serotonin type 3 5HT₃ receptor (Hassaine et al., 2014), the GABA_{A} receptor (Masiulis et al., 2019), and the bacterial ELIC (Brams et al., 2020). Thus, a need exists in the art for small molecules specifically targeting α7-nAChRs. The invention fulfills this need.

### BRIEF SUMMARY OF THE INVENTION

The invention encompasses compositions and methods for producing and using antibodies, more specifically single domain antibodies (sdAbs) (for instance from camelids or sharks) and even more specifically to VHH (“nanobody”), against alpha7 nAChR. In one embodiment, the invention encompasses an isolated antibody that is an allosteric modulator of alpha7 nAChR.

The amino acid sequence of alpha7 nAChR is the following:

(SEQ ID NO: 57)
MRCSPGGVWLALAASLLHVSLOGEFORKLYKELVKNYNPLERPVANDSQ
PLTVYFSLSLLQIMDVDEKNQVLTTNIWLQMSWTDHYLQWNVSEYPGVK
TVRFPDGQIWKPDILLYNSADERFDATFHTNVLVNSSGHCQYLPPGIFK
SSCYIDVRWFPFDVQHCKLKFGSWSYGGWSLDLQMQEADISGYIPNGEW
DLVGIPGKRSERFYECCKEPYPDVTFTVTMRRRTLYYGLNLLIPCVLIS
ALALLVELLPADSGEKISLGITVLLSLTVEMLLVAEIMPATSDSVPLIA
QYFASTMIIVGLSVVVTVIVLQYHHHDPDGGKMPKWTRVILLNWCAWFL
RMKRPGEDKVRPACQHKQRRCSLASVEMSAVAPPPASNGNLLYIGFRGL
DGVHCVPTPDSGVVCGRMACSPTHDEHLLHGGQPPEGDPDLAKILEEVR
YIANRFRCQDESEAVCSEWKFAACVVDRLCLMAFSVFTIICTIGILMSA
PNFVEAVSKDFA

In one embodiment, the invention encompasses an isolated antibody that binds to an epitope comprising residues of the sequence of the alpha7 nAChR selected in the group consisting of residues 24, 27-28, 31, 85-86, 88, 90 and 93 of the alpha7 nAChR (FIG. 17). In one embodiment, the invention encompasses an isolated antibody that binds to an epitope comprising residues of the sequence of the alpha7 nAChR selected in the group consisting of residues 24, 27-28, 31, 46 and/or the mannose grafted on said residue, 85-86, 88, 90 and 93 of the alpha7 nAChR (FIG. 17).

In one embodiment, the invention encompasses an isolated antibody that binds to an epitope comprising residues of the sequence of the alpha7 nAChR selected in the group consisting of residues 24, 27-28, 31-32, 35-36, 46, 85-86, 88, 93 of the alpha7 nAChR (FIG. 17). In one embodiment, the invention encompasses an isolated antibody that binds to an epitope comprising residues of the sequence of the alpha7 nAChR selected in the group consisting of residues 24, 27-28, 31-32, 35-36, 46 and/or the mannose grafted on said residue, 85-86, 88, 93 of the alpha7 nAChR (FIG. 17).

In one embodiment, the antibody is or comprises a single domain antibody, preferably a VHH (also named nanobody).

In one embodiment, the single domain antibody, preferably the VHH, comprises a CDR1 having the amino acid sequence SGFTFAHYAMV (SEQ ID NO: 18) or SGGTFSHYAVG (SEQ ID NO: 19) or XGXTFXHYAXX (SEQ ID NO: 14) wherein X means an undefined amino acid. In one embodiment, the single domain antibody, preferably the VHH comprises a CDR1 having the amino acid sequence SGGTFSSYAIG (SEQ ID NO: 16), SGRTVGTYTMG (SEQ ID NO: 17), SGFTLDYYTIG (SEQ ID NO: 20), PGITLSRYGMYGMG (SEQ ID NO: 21), or SGRTFSSYSM (SEQ ID NO: 22).

In one embodiment, the single domain antibody, preferably, the VHH, comprises a CDR2 having the amino acid sequence GISWSGASTYYAS (SEQ ID NO: 28) or AISWSGRSTSFAN (SEQ ID NO: 29) or XISWSGXSTXXAX (SEQ ID NO: 24) wherein X means an undefined amino acid. In one embodiment, the single domain antibody, preferably the VHH comprises a CDR2 having the amino acid sequence AISWSGVSTDYAG (SEQ ID NO: 26), SISGAVGTTYYAD (SEQ ID NO: 27), CIRGSGGSTNYAD (SEQ ID NO: 30), AITWSGGQTYYQD (SEQ ID NO: 31), or AINWSGGTTYYAD (SEQ ID NO: 32).

In one embodiment, the single domain antibody, preferably the VHH, comprises a CDR3 having the amino acid sequence AAARFGVGVDDDYSY (SEQ ID NO: 35) or APARFGTGSAARDEYDD (SEQ ID NO: 36). In one embodiment, the single domain antibody, preferably the VHH, comprises a CDR3 having the amino acid sequence AAARFGTSSPDDEYHY (SEQ ID NO: 33), AAGSFPLTRTNYVQF (SEQ ID NO: 34), AADFLSTCSLAGYRYEEV (SEQ ID NO: 37), AADGDRFYPEPVVDDNAYKF (SEQ ID NO: 38), or AAGGTTAQGMSVMTPRLGS (SEQ ID NO: 39).

In one embodiment, the single domain antibody, preferably the VHH, comprises the amino acid sequence of VHH α7E3 (SEQ ID NO: 4) or VHH α7C4 (SEQ ID NO: 3). In one embodiment, the single domain antibody, preferably the VHH consists of the amino acid sequence of VHH α7E3 (SEQ ID NO: 4) or VHH α7C4 (SEQ ID NO: 3).

In one embodiment, the single domain antibody, preferably the VHH, comprises the following complementary determining regions (CDR):
CDR1 having the amino acid sequence selected from SEQ ID NO: 13-22 and variants thereof having no more than 2 mismatches compared to SEQ ID NO: 13-22;
CDR2 having the amino acid sequence selected from SEQ ID NO: 23-32 and variants thereof having no more than 2 mismatches compared to SEQ ID NO: 23-32; and
CDR3 having the amino acid sequence selected from SEQ ID NO: 33-39 and variants thereof having no more than 2 mismatches compared to SEQ ID NO: 33-39.

In one embodiment, the VHH consists of the amino acid sequence of VHH α7E3 (SEQ ID NO: 4) and binds to an epitope comprising residues of the sequence of the alpha7 nAChR selected from the group consisting of residues 24, 27-28, 31, 46, 85-86, 88, 90 and 93 of the alpha7 nAChR (SEQ ID NO: 57). In one embodiment, the VHH consists of the amino acid sequence of VHH α7C4 (SEQ ID NO: 3) and binds to an epitope comprising residues of the sequence of the alpha7 nAChR (SEQ ID NO: 57) selected from the group consisting of residues 24, 27-28, 31, 85-86, 88, 90 and 93 of the alpha7 nAChR (SEQ ID NO: 57).

In one embodiment, the VHH consists of the amino acid sequence of VHH α7E3 (SEQ ID NO: 4) and binds to an epitope comprising residues of the sequence of the alpha7 nAChR selected from the group consisting of residues 24, 27-28, 31-32, 35-36, 46, 85-86, 88, 93 of the alpha7 nAChR (SEQ ID NO: 57). In one embodiment, the VHH E3 binds to an epitope comprising residues of the sequence of the alpha7 nAChR selected in the group consisting of residues 24, 27-28, 31-32, 35-36, 46 and/or the mannose grafted on said residue, 85-86, 88, 93 of the alpha7 nAChR (SEQ ID NO: 57).

In one embodiment, the VHH consists of the amino acid sequence of VHH α7C4 (SEQ ID NO: 3) and binds to an epitope comprising residues of the sequence of the alpha7 nAChR (SEQ ID NO: 57) selected from the group consisting of residues 24, 27-28, 31-32, 35-36, 46, 85-86, 88, 93 of the alpha7 nAChR (SEQ ID NO: 57). In one embodiment, the VHH C4 binds to an epitope comprising residues of the sequence of the alpha7 nAChR selected in the group consisting of residues 24, 27-28, 31-32, 35-36, 46 and/or the mannose grafted on said residue, 85-86, 88, 93 of the alpha7 nAChR (SEQ ID NO: 57).

In one embodiment, the antibody is a multimeric construct comprising the sdAb of the invention, preferably VHH of the invention covalently linked to at least one second polypeptide. The at least one second polypeptide may be a sdAb, preferably a VHH or not.

In one embodiment, the antibody is monovalent.

In one embodiment, the antibody is multivalent, preferably bivalent.

In one embodiment, the antibody is engineered to cross the blood-brain barrier.

In one embodiment, the antibody is a fusion between the VHH of the invention and a second VHH targeting the transferrin receptor.

In one embodiment, the invention encompasses a nucleic acid encoding the antibody of the invention.

In one embodiment, the invention encompasses a vector comprising a nucleic acid of the invention.

The present invention also relates to a composition comprising the antibody of the invention and a pharmaceutically acceptable vehicle.

In one embodiment, the invention encompasses a method comprising administering the antibody of the invention or the composition of the invention.

In one embodiment, the invention encompasses the use of the antibody of the invention or of the composition of the invention to treat cognitive disorders.

In one embodiment, the invention encompasses the use of the antibody of the invention or of the composition according to the invention to treat a disease selected from the group consisting of Alzheimer's disease, Parkinson's disease and schizophrenia.

In one embodiment, the antibody of the invention or the composition of the invention is for use in a method of treatment.

In one embodiment, the antibody of the invention or the composition of the invention is for use in a method of treatment of cognitive disorders.

In one embodiment, the antibody of the invention or the composition of the invention is for use in a method of treatment of a disease selected from the group consisting of Alzheimer's disease, Parkinson's disease and schizophrenia.

Another subject matter of the invention relates to a detection agent comprising the antibody of the invention and a label.

Another subject matter of the invention relates to a method for the detection of alpha7 nAChR comprising the steps of:
providing a detection agent comprising an antibody of the invention;
providing a biological sample;
contacting the detection agent with the biological sample; and
visualizing the antigen-detection agent complexes formed.

### BRIEF DESCRIPTION OF THE DRAWINGS

FIG. 1: VHH α7E3 and α7C4 with Related Constructs. Sequence alignment of α7E3 and α7C4 VHHs with numbering starting after the signal peptide, although sometimes incomplete cleavage occurs, and the preceding MA is left attached. The Myc tag is highlighted with gray shading, 6×His tag is underlined, and each CDR is boxed. N- and C-terminal changes for the Fc, CSA, and bivalent constructs are also shown.

FIG. 2: VHH α7E3 and α7C4 with Related Constructs. Structural representation of each variation with the tags labeled and their side-chains represented as sticks, the N- and C-terminals labeled, as well as the CDRs.

FIG. 3a-c: VHH characterization by immunofluorescence

Dapi stains the cells' nucleus. Images are representative of at least n=4.
A. Merged images (Dapi) of CHO cells left: non-transfected; right: expressing hα7-nAChR; immunostained with 1 μg/mL of conjugated α7E3-Alexa488.
B. Images (Dapi, GFP, Alexa647, Merged) of non-permeabilized HEK 293 cells expressing hα7-nAChR immunostained with α7E3-Fc and α7C4-Fc, demonstrating an extracellular binding. Cytoplasmic GFP indicates efficiently transfected cells.
C. Images (Dapi, GFP, Alexa647, Merged) of permeabilized HEK 293 cells expressing α7-, α3β4_{StrepII}- and α4_{GFP}β2-nAChRs immunostained using α7E3-Fc (left) and α7C4-Fc (right). For α7 and α3B4_{Strep}, cytoplasmic GFP indicates efficiently transfected cells. The VHHs were detected by an anti-human IgG coupled to Alexa647 (red). Identical exposure times were used to visualize each channel on all conditions.

FIG. 4: α7E3-VHH Characterization by TEVC Electrophysiology. Representative concentration dependent traces with indicated concentrations of VHH α7E3 by oocytes injected with a hα7-IRES-NACHO plasmid. Traces are all on the same oocyte and applied chronologically in the same order as listed. “Post-perfusion” protocol with α7E3 co-applied in the middle of a 30 μM ACh response. (representative of at least n=4)

FIG. 5: α7E3-VHH Characterization by TEVC Electrophysiology “Co-perfusion” protocol with α7E3 co-applied for the same duration as 30 μM ACh (representative of at least n=4). 30 μM ACh response without E3 (0 nM) at the beginning and end of the dose-response curve show a complete wash of α7E3 and stable response of the oocyte.

FIG. 6: α7E3-VHH Characterization by TEVC Electrophysiology “Pre-perfusion” protocol using 60 s pre-perfusion of α7E3 (entire time is not shown, the baseline stays flat the entire time) with the concentrations of E3 indicated. 30 μM ACh response without E3 (0 nM) at the beginning and end of the dose-response curve show a complete wash of α7E3 and stable response of the oocyte.

FIG. 7: α7E3-VHH Characterization by TEVC Electrophysiology: Percent potentiation of 30 μM ACh peak response (base) by various pre-perfusion times of VHH α7E3 in a dose-dependent manner (n=5 over 2 oocytes). Maximum potentiation is achieved by 60 s pre-perfusion with 30 s producing only a slightly smaller potentiation, therefore it was decided to keep a 30 s pre-perfusion for subsequent experiments.

FIG. 8: α7E3-VHH Characterization by TEVC Electrophysiology: pre-application of mixture of α7E3 and α7C4 abolishes the potentiation shown with α7E3 alone, indicating that α7E3 and α7C4 display overlapping binding sites on the receptor structure.

FIG. 9: I¹²⁵-αBtx Competition Binding to hα7/m5-HT₃A. Scintillation counts per minute (CPM) of 25 nM I¹²⁵-αBtx bound to hα7/m5-HT₃A transiently transfected HEK293 cells in competition with 1.11 mM ACh or ˜300 nM VHH. Mean of n=2 of triplicate reads, with error bars showing the standard deviation of the mean.

FIG. 10: α7E3E3 bivalent VHH characterization by TEVC Electrophysiology. Representative (of n=11) overlay of concentration dependent traces with concentrations of α7E3E3 bivalent VHH indicated using a “purely pre-perfusion” protocol.

FIG. 11: α7E3E3 bivalent characterization by TEVC Electrophysiology. An extension of the same traces from the FIG. 10 shown in a chronological fashion, where additional applications of 30 μM ACh alone (not shown on FIG. 10) before and after the concentration dependent application of α7E3E3 bivalent VHH are included (traces within the grey box). Data show that potentiation is maintained even 40 min after last application of the bivalent α7E3E3.

FIG. 12: ACh affinity in presence of α7E3 and α7E3E3. ACh concentration response curves without and using a 30 s “pre-perfusion” protocol with a fixed concentration of α7E3 and bivalent α7E3E3, where effectively the oocyte is in a constant presence of the bivalent variation. Data show that ACh affinity (indicated in the box) is not significantly altered by either α7E3 or the bivalent α7E3E3.

FIG. 13: Immunofluorescence expression controls

Representative images of HEK 293 cells expressing hα7- (upper) and hα3 hβ4_{StrepII}- (lower) nAChRs immunostained using conjugated α-Bgt-Alexa647 and an anti-StrepII tag detected by a conjugated anti-mouse IgG-Alexa647 respectively. Dapi stains the cells' nucleus. Cytoplasmic eGFP indicates efficiently transfected cells. Identical exposure times were used to visualize each channel on all conditions.

FIG. 14: VHH sequences of the 7 generated VHHs binding to the alpha7 receptor. The CDRs are in bold.

FIG. 15: Single particle cryo-electron microscopy structures of α7E3 in complex with the human α7 receptor (in the presence of nicotine). α7E3 binds to its epitope at the extracellular top of the receptor. α7E3 binds at the interface between two subunits, and five VHHs are bound per pentameric receptor. Lower panel: zoom on the VHH/receptor interaction, showing that the epitope includes different portions of the N-terminal helix of two subunits, as well as an extended loop called “MIR loop”. It was shown by immunofluorescence assay that the mutation of the MIR loop abolishes α7E3 binding (data not represented).

FIG. 16: Single particle cryo-electron microscopy structures of α7C4 in complex with the human α7 receptor (in the presence of nicotine). α7E3 and α7C4 bind to the same epitope at the extracellular top of the receptor. α7E3 and α7C4 bind at the interface between two subunits, and five VHHs are bound per pentameric receptor. It was shown by immunofluorescence assay that the mutation of the MIR loop abolishes α7C4 binding (data not represented)

FIG. 17: Amino acid sequence of the human α7 receptor, coloring in bold the residues contributing to the E3 VHH epitope, with the N-terminal helix (full line) and the “MIR” loop (dotted line) underlined. Highlighted in bold and italics is an additional residue comprising a mannose grafted and contributing to the E3 VHH epitope.

FIG. 18: passage of nanobody E3 across the blood-brain barrier at different time points after nanobody perfusion on microfluidic devices containing a semi-permeable membrane where confluent mice brain endothelial cells are grown.

### DETAILED DESCRIPTION OF THE INVENTION

The generation and functional characterization of VHHs specifically targeting the alpha7 nAChR is described. Seven single-domain antibody fragments of camelids were generated. They were generated through immunization of an alpaca with cells expressing the extracellular domain of the human α7-nAChR and selected to bind the α7-nAChR. Among them, two VHHs, named α7E3 and α7C4, were analyzed in detail. Immunofluorescence assays show that they bind to the α7-nAChR but not to the other major nAChR subtypes α4β2 and α3β4. Two-electrode voltage clamp electrophysiology shows that α7E3 acts as a slowly associating type I PAM, strongly potentiating the ACh-elicited currents mainly at ACh concentrations bellow its EC₅₀, while not significantly altering the desensitization of the receptor. An α7E3E3 bivalent construct shows similar potentiating properties but displays very slow dissociation kinetics conferring quasi-irreversible properties. α7C4 does not alter the receptor function, but fully inhibits the α7E3-evoked potentiation, showing it is a silent allosteric modulator competing with α7E3 binding. Both VHHs are not competing with the binding of α-bungarotoxin, showing a binding site location away from the acetylcholine binding site. In conclusion, the two VHHs α7C4 and α7E3 constitute a novel class of allosteric modulators of the α7-nAChR, that will be useful for pharmacological and structural investigations, with potential clinical applications.

The classical technique to generate specific VHHs against a given target is to immunize alpacas with purified protein, a procedure that requires large quantities of protein (>1 mg). Within the pLGIC family, this technique has been successfully applied for members yielding good expression in recombinant systems such as the ELIC (Brams et al., 2020), the 5-HT₃ receptor (Hassaine et al., 2014), and the GABA_{A} receptor (Masiulis et al., 2019).

In contrast, the α7-nAChR is a subtype that shows low levels of expression in recombinant systems. To avoid the time-consuming overexpression and purification steps, direct immunization with a cell-line transiently transfected with a hα7-nAChR/5-HT₃A chimera that has good expression levels was performed. Such a procedure is expected to stimulate the production of a wide range of VHHs recognizing many proteins present at the surface of the cells. Therefore, a carefully designed panning strategy was completed, yielding, after a few rounds, two VHHs that bind specifically to the α7-nAChR. The procedure has also the advantage of injecting membrane-inserted protein, ensuring native pentameric assembly of the receptor, increasing the chance to isolate antibodies targeting a properly folded ECD in a pentameric conformation. This procedure should be applicable to other pLGICs with weak expression levels and/or low stability after extraction from the plasma membrane.

The PAM properties of α7E3 suggest that the VHH binds with higher affinity to the active state as compared to the resting state of the α7-nAChR. Interestingly, the α7-nAChR is activated by choline with an EC₅₀ around 500 μM in an oocyte, with choline already producing clearly detectable responses at concentrations of 30 μM or less (Papke and Porter Papke, 2002). Since levels of choline in human plasma have been reported to range between 5 to 15 μM (Buchman et al 2000), it is possible that such a concentration in the alpaca plasma would stabilize a significant fraction of the receptor on the cells which were injected in the active or desensitized conformational states during immunization to direct the generation of a PAM. In addition, the type I PAM character of α7E3 suggest that the VHH binds with similar affinities to the active and desensitized state, thereby not impairing the kinetics and extent of the desensitization process. This assessment is in line with the recently published cryo-EM structures of the receptor in nanodiscs, that suggest that the main structural reorganizations associated with active to desensitized transition are located in the TMD, with weak reorganization in the ECD (Noviello et al., 2021).

Interestingly, the dimerization of α7E3 allows the formation of a quasi-irreversible type I PAM. This striking effect is probably related to an increase of the avidity of this bivalent molecule. In retrospect, this feature probably explains why, in immunofluorescence experiments that involves several rinsing steps after VHH binding, the labeling by monovalent α7E3 was weak, while that of bivalent α7E3-Fc was robust. Several examples of dimerization of VHHs directed against viral proteins have been reported to potentiate the neutralizing activities of the parental VHH (Hultberg et al., 2011) (Dong et al., 2020) (Schoof et al., 2020) (Terryn et al., 2014).

In conclusion, the two VHHs α7C4 and α7E3 constitute a novel class of allosteric modulators of the α7-nAChR. They show high specificity among nAChR subtypes, a feature characteristic of antibody antigen recognition that involve a large area of interaction. These VHHs are easily expressed in milligram amounts in cell-lines and can be easily engineered as illustrated here by the generation of a quasi-irreversible bivalent potentiator. They will be useful for a wide range of applications, notably the investigation of native receptors in brain tissues in immunofluorescence and immunoprecipitation assays. They will be also precious for the investigation of the receptor molecules and as pharmacological tools to help structural studies. Finally, they constitute an original family of allosteric modulators with potential applications such as cognitive enhancers, or as a potential treatment against α7-nAChR auto-antibodies that are found in some patients diagnosed with schizophrenia (Chandley et al., 2009).

### A. Methods for Generating Antibodies

The invention encompasses the generation of antibodies, more specifically single domain antibodies (for instance from camelids or sharks) and even more specifically to VHH, against α7-nAChR. Antibodies (e.g., VHH) can be made by routine techniques in the art. For example, alpacas can be immunized (see, e.g., Brams et al., 2020; Hassaine et al., 2014; Masiulis et al., 2019; Gransagne et al., 2022).

Preferably, alpacas are immunized by direct immunization with a cell-line transiently transfected with a nAChR/5HT₃ chimera (e.g., hα7-nAChR/5-HT₃A) that has good expression levels. Such a procedure is expected to stimulate the production of a wide range of VHHs recognizing many proteins present at the surface of the cells.

In some embodiments, an alpaca is immunized and a library containing different VHHs is constructed from cDNA encoding VHH domains isolated from lymphocytes (see, e.g., Gransagne et al., 2022). VHHs can then be selected by phage display through a panning strategy, such as that used in the examples, to select VHHs that bind specifically to the α7-nAChR.

The DNA of the phage encoding the VHH can be inserted into an expression vector. Thus, the invention encompasses expression vectors encoding the VHH of the invention, cells containing these expression vectors, methods of producing antibodies by introduction of the expression vectors into cells and expression of the vector, and the antibodies produced.

### B. Antibodies

The invention encompasses an isolated antibody that is an allosteric modulator of alpha7 nAChR. In some embodiments, the antibody is a silent allosteric modulator (SAM). In some embodiments, the antibody is a positive allosteric modulator (PAM).

In some embodiments, the invention encompasses an isolated antibody that binds to an epitope comprising residues of the sequence of the alpha7 nAChR selected from the group consisting of residues 24, 27-28, 31, 85-86, 88, 90 and 93 of the alpha7 nAChR (FIG. 17). In some embodiments, the invention encompasses an isolated antibody that binds to an epitope comprising residues of the sequence of the alpha7 nAChR selected from the group consisting of residues 24, 27-28, 31, 46 and/or the mannose grafted on said residue, 85-86, 88, 90 and 93 of the alpha7 nAChR (FIG. 17).

In some embodiments, the invention is a single domain antibody.

In preferred embodiments, the antibody is a VHH (also named herein nanobody). A VHH is the variable domain of a heavy-chain-only antibody from a camelid (HcAb) or a molecule derived from such a VHH and having substantially the same properties as the original VHH in particular in respect of antigen recognition capacity (including when having no antigen recognition capacity). All the species of the Camelidea family have heavy-chain-only antibodies. In a preferred embodiment, the VHH of the invention is obtained from an alpaca (Lama pacos).

In various embodiments, the VHH of the invention has one of the sequences shown in FIG. 14. In various embodiments, the VHH of the invention are α7E3 or α7C4.

The VHH may also have a variant sequence having at least 70% or at least 80% identity, preferably at least 90% identity, more preferably at least 95% identity and even more preferably at least 99% identity with said sequence. If the VHH of the invention comprises only a portion of the sequence, the identity level is calculated on the sequence of said portion. The length of said portion is at least 70%, preferably at least 80%, more preferably at least 90% and even more preferably at least 95% of the length of the full VHH.

In preferred embodiments, the length of said portion is at least 60 amino acids, at least 80 amino acids, at least 100 amino acids, at least 110 amino acids, at least 120 amino acids, at least 130 amino acids, at least 140 amino acids, at least 150 amino acids. In particular embodiments, the VHH of the invention comprises three CDR regions, said CDR regions being selected from the group consisting of the CDR regions with the sequence of 1, 2, or 3 of the CDRs in FIG. 14.

In an embodiment, the VHH of the invention comprises or consists of the amino acid sequence selected from the group consisting of SEQ ID NO: 1-12 or is a variant thereof having at least 70% or at least 80% identity, preferably at least 90% identity, more preferably at least 95% identity and even more preferably at least 99% identity with said sequence.

In an embodiment, the VHH comprises a CDR1 having the amino acid sequence selected from the group consisting of SEQ ID NO: 13-22.

The amino acid sequence SEQ ID NO: 13 is the following:
SGX₁TX₂X₃X₄YX₅X₆X₇ with X₁ is F, G or R, X₂ is L, V or F, X₃ is S, G, A or D, X₄ is S, T, H or Y, X₅ is A, T or S, X₆ is I, M or V and X₇ is M or G.

The amino acid sequence SEQ ID NO: 14 is the following:
XGXTFXHYAXX with X is an undefined amino acid.

The amino acid sequence SEQ ID NO: 15 is the following:
SGX₁TFX 2HYAX₃X₄ with X₁ is F or G, X₂ is A or S, X₃ is M or V and X₄ is M or G.

In various embodiments, the VHH comprises a CDR1 having the amino acid sequence SGFTFAHYAMV (SEQ ID NO: 18) or SGGTFSHYAVG (SEQ ID NO: 19) or XGXTFXHYAXX (SEQ ID NO: 14).

In an embodiment, the VHH comprises a CDR2 having the amino acid sequence selected from the group consisting of SEQ ID NO: 23-32.

The amino acid sequence SEQ ID NO: 23 is the following:
X₁IX₂WSGX₃X₄TX₅X₆X₇X₈ with X₁ is G or A, X₂ is T, S or N, X₃ is A, V, G or R, X₄ is S, Q or T, X₅ is D, Y or S, X₆ is Y or F, X₇ is Q or A and X₈ is G, D S or N.

The amino acid sequence SEQ ID NO: 24 is the following:
XISWSGXSTXXAX wherein X is an undefined amino acid.

The amino acid sequence SEQ ID NO: 25 is the following:
X₁ISWSGX₂STX₃X₄AX₅ with X₁ is G or A, X₂ is A or R, X₃ is Y or S, X₄ is Y or F and X₅ is S or N.

In various embodiments, the VHH comprises a CDR2 having the amino acid sequence

(SEQ ID NO: 28)
GISWSGASTYYAS
or
(SEQ ID NO: 29)
AISWSGRSTSFAN
or
(SEQ ID NO: 24)
XISWSGXSTSSAX.

In an embodiment, the VHH comprises a CDR2 having the amino acid sequence selected from the group consisting of SEQ ID NO: 33-39.

In various embodiments, the VHH comprises a CDR3 having the amino acid sequence

(SEQ ID NO: 35)
AAARFGVGVDDDYSY
or
(SEQ ID NO: 36)
APARFGTGSAARDEYDD.

In one embodiment, the VHH comprises a CDR1 having the amino acid sequence SGFTFAHYAMV (SEQ ID NO: 18) or SGGTFSHYAVG (SEQ ID NO: 19), a CDR2 having the amino acid sequence GISWSGASTYYAS (SEQ ID NO: 28) or AISWSGRSTSFAN (SEQ ID NO: 29), and a CDR3 having the amino acid sequence AAARFGVGVDDDYSY (SEQ ID NO: 35) or APARFGTGSAARDEYDD (SEQ ID NO: 36).

In one embodiment, the VHH comprises a CDR1 having the amino acid sequence SGFTFAHYAMV (SEQ ID NO: 18), a CDR2 having the amino acid sequence GISWSGASTYYAS (SEQ ID NO: 28), and a CDR3 having the amino acid sequence AAARFGVGVDDDYSY (SEQ ID NO: 35).

In one embodiment, the VHH comprises a CDR1 having the amino acid sequence SGGTFSHYAVG (SEQ ID NO: 19), a CDR2 having the amino acid sequence AISWSGRSTSFAN (SEQ ID NO: 29), and a CDR3 having the amino acid sequence APARFGTGSAARDEYDD (SEQ ID NO: 36).

In one embodiment, the VHH comprises a CDR1 having the amino acid sequence SGGTFSSYAIG (SEQ ID NO: 16), a CDR2 having the amino acid sequence AISWSGVSTDYAG (SEQ ID NO: 26), and a CDR3 having the amino acid sequence AAARFGTSSPDDEYHY (SEQ ID NO: 33).

In one embodiment, the VHH comprises a CDR1 having the amino acid sequence SGRTVGTYTMG (SEQ ID NO: 17), a CDR2 having the amino acid sequence SISGAVGTTYYAD (SEQ ID NO: 27), and a CDR3 having the amino acid sequence AAGSFPLTRTNYVQF (SEQ ID NO: 34).

In one embodiment, the VHH comprises a CDR1 having the amino acid sequence SGFTLDYYTIG (SEQ ID NO: 20), a CDR2 having the amino acid sequence CIRGSGGSTNYAD (SEQ ID NO: 30), and a CDR3 having the amino acid sequence AADFLSTCSLAGYRYEEV (SEQ ID NO: 37).

In one embodiment, the VHH comprises a CDR1 having the amino acid sequence PGITLSRYGMYGMG (SEQ ID NO: 21), a CDR2 having the amino acid sequence AITWSGGQTYYQD (SEQ ID NO: 31), and a CDR3 having the amino acid sequence AADGDRFYPEPVVDDNAYKF (SEQ ID NO: 38).

In one embodiment, the VHH comprises a CDR1 having the amino acid sequence SGRTFSSYSMG (SEQ ID NO: 22), a CDR2 having the amino acid sequence AINWSGGTTYYAD (SEQ ID NO: 32), and a CDR3 having the amino acid sequence AAGGTTAQGMSVMTPRLGS (SEQ ID NO: 39).

In various embodiments, the VHH comprises a CDR1 having the amino acid sequence XGXTFXHYAXX (SEQ ID NO: 14) and a CDR2 having the amino acid sequence XISWSGXSTXXAX (SEQ ID NO: 24).

In various embodiments, the VHH comprises a CDR3 having the amino acid sequence

(SEQ ID NO: 35)
APARFGVGVDDDYSY
or
(SEQ ID NO: 36)
AAARFGTGSAARFEDYY.

In one embodiment, the VHH comprises a CDR1 having the amino acid sequence

(SEQ ID NO: 16)
SGGTFSSYAIG,
(SEQ ID NO: 17)
SGRTVGTYTMG,
(SEQ ID NO: 20)
SGFTLDYYTIG,
(SEQ ID NO: 21)
PGITLSRYGMYGMG,
or
(SEQ ID NO: 22)
SGRTFSSYSM.

In one embodiment, the VHH comprises a CDR2 having the amino acid sequence

(SEQ ID NO: 26)
AISWSGVSTDYAG,
(SEQ ID NO: 27)
SISGAVGTTYYAD,
(SEQ ID NO: 30)
CIRGSGGSTNYAD,
(SEQ ID NO: 31)
AITWSGGQTYYQD,
or
(SEQ ID NO: 32)
AINWSGGTTYYAD.

In one embodiment, the VHH comprises a CDR3 having the amino acid sequence

(SEQ ID NO: 33)
AAARFGTSSPDDEYHY,
(SEQ ID NO: 34)
AAGSFPLATRTNYVQF,
(SEQ ID NO: 37)
AADFLSTVSLAGYRYEEV,
(SEQ ID NO: 38)
AADGDRFYPEPVVDDNAYKF,
or
(SEQ ID NO: 39)
AAGGTTAQGMSVMTPRLGS.

In various embodiment, the VHH consists of or comprises any of the following amino acid sequences or any of the sequences in FIG. 14. The complementary determining regions (CDR) and framework regions (FW) are summarized in the tables below:

| VHH | B7 | C3 | C4 | |
|---|---|---|---|---|
| FW1 | EVQLQASGGGLVQAGGSLRL | DVQLVESGGGLVQTGGSLRVSCAP | QVQLVESGGGLVQAGGSLKLS | |
| | SCAA | (SEQ ID NO. 73) | CAA | |
| | (SEQ ID NO. 72) | | (SEQ ID NO. 74) | |
| CDR1 | SGGTFSSYAIG | SGRTVGTYTMG | SGFTFAHYAMV | |
| | (SEQ ID NO. 16) | (SEQ ID NO. 17) | (SEQ ID NO. 18) | |
| FW2 | WFRQTPGKEREFVA | WFRQAPGENRDFVA | WFRQAPGKEREFVA | |
| | (SEQ ID NO. 79) | (SEQ ID NO. 80) | (SEQ ID NO. 81) | |
| CDR2 | AISWSGVSTDYAG | SISGAVGTTYYAD | GISWSGASTYYAS | |
| | (SEQ ID NO. 26) | (SEQ ID NO. 27) | (SEQ ID NO. 28) | |
| FW3 | SVKGRFTI SRDNAKNTVYLQ | SVQGRFTISRDNAKNTVYLQMNSL | SVKGRFTISRDNAKNTVYLQM | |
| | MNSLKPEDTAVYYC | KPEDTAVYYC | NSLKPEDTAVYYV | |
| | (SEQ ID NO. 86) | (SEQ ID NO. 87) | (SEQ ID NO. 88) | |
| CDR3 | AAARFGTSSPDDEYHY | AAGSFPLTRTNYVQF | AAARFGVGVDDDYSY | |
| | (SEQ ID NO. 33) | (SEQ ID NO. 34) | (SEQ ID NO. 35) | |
| FW4 | WGHGTQVTVSS | WGQGTQVTVSS | WGQGTQVTVSS | |
| | (SEQ ID NO. 93) | (SEQ ID NO. 94) | (SEQ ID NO. 95) | |
| VHH | E3 | D6 | F1 | F5 |
| FW1 | EVQLQASGGGLVQAG | QLQLVESGGGLVQPGGS | EVQLVESGGGLVQAG | DVQLVESGGGLVQAG |
| | DSLRLSCAA | LRLSCAA | GSLRLSCAA | GSLRLSCAA |
| | (SEQ ID NO. 75) | (SEQ ID NO. 76) | (SEQ ID NO. 77) | (SEQ ID NO. 78) |
| CDR1 | SGGTFSHYAVG | SGFTLDYYTIG | PGITLSRYGMYGMG | SGRTFSSYSMG |
| | (SEQ ID NO. 19) | (SEQ ID NO. 20) | (SEQ ID NO. 21) | (SEQ ID NO. 22) |
| FW2 | WFRQAPGKEREFVA | WFRQAPGKEREGVS | WFRQAPGKEREFVA | WFRQAPGKEREFVA |
| | (SEQ ID NO. 82) | (SEQ ID NO. 83) | (SEQ ID NO. 84) | (SEQ ID NO. 85) |
| CDR2 | AISWSGRSTSFAN | CIRGSGGSTNYAD | AITWSGGQTYYQ | AINWSGGTTYYAD |
| | (SEQ ID NO. 29) | (SEQ ID NO. 30) | (SEQ ID NO. 31) | (SEQ ID NO. 32) |
| FW3 | SVKGRFTISRDSAKN | SVKGRFTISRDNAKNTV | SVKGRFTISRDNAKK | SVKGRFTISRDNAKN |
| | TAYLQMNNLKPEDTA | YLQMNSLKPEDTAVYYC | LTFLQMNSLKPEDTA | TVYLQMNSLKPEDTA |
| | VYCC | (SEQ ID NO. 90) | VYY | VYYC |
| | (SEQ ID NO. 89) | | (SEQ ID NO. 91) | (SEQ ID NO. 92) |
| CDR3 | APARFGTGSAARDEYDD | AADFLSTCSLAGYRYEEV | AADGDRFYPEPVVDD | AAGGTTAQGMSVMTP |
| | (SEQ ID NO. 36) | (SEQ ID NO. 37) | NAYKF | RLGS |
| | | | (SEQ ID NO. 38) | (SEQ ID NO. 39) |
| FW4 | CGQGTQVTVSS | WGQGTLVTVSS | WGQGTQVTVSS | WGQGTQVTVSS |
| | (SEQ ID NO. 96) | (SEQ ID NO. 97) | (SEQ ID NO. 98) | (SEQ ID NO. 99) |

> α7E3 (also named E3) (deposited on Nov. 8, 2022
at the Collection Nationale de Cultures des
Microorganismes, 25 rue du Docteur Roux, 75015
Paris, France under No. CNCM 1-5916).
(SEQ ID NO: 4)
MAEVQLQASGGGLVQAGDSLRLSCAASGGTFSHYAVGWFRQAPGKEREF
VAAISWSGRSTSFANSVKGRFTISRDSAKNTAYLQMNNLKPEDTAVYCC
APARFGTGSAARDEYDDCGQGTQVTVSS;
> α7C4 (also named C4) (deposited on Nov. 8, 2022
at the Collection Nationale de Cultures des
Microorganismes, 25 rue du Docteur Roux, 75015
Paris, France under No. CNCM 1-5915).
(SEQ ID NO: 3)
MAQVQLVESGGGLVQAGGSLKLSCAASGFTFAHYAMVWFRQAPGKEREF
VAGISWSGASTYYASSVKGRFTISRDNAKNTVYLQMNSLKPEDTAVYYV
AAARFGVGVDDDYSYWGQGTQVTVSS;
> α7B7 (also named B7)
(SEQ ID NO: 1)
MAEVQLQASGGGLVQAGGSLRLSCAASGGTFSSYAIGWFRQTPGKEREF
VAAISWSGVSTDYAGSVKGRFTISRDNAKNTVYLQMNSLKPEDTAVYYC
AAARFGTSSPDDEYHYWGHGTQVTVSS;
> α7C3 (also named C3)
(SEQ ID NO: 2)
MADVQLVESGGGLVQTGGSLRVSCAPSGRTVGTYTMGWFRQAPGENRDF
VASISGAVGTTYYADSVQGRFTISRDNAKNTVYLQMNSLKPEDTAVYYC
AAGSFPLTRINYVQFWGQGTQVTVSS;
> α7E6 (also named E6)
(SEQ ID NO: 10)
MAQLQLVESGGGLVQPGGSLRLSCAASGFTLDYYTIGWFRQAPGKEREG
VSCIRGSGGSTNYADSVKGRFTISRDNAKNTVYLQMNSLKPEDTAVYYC
AADFLSTCSLAGYRYEEVWGQGTLVTVSS;
> α7F1 (also named F1)
(SEQ ID NO: 11)
MAEVQLVESGGGLVQAGGSLRLSCAAPGITLSRYGMYGMGWFRQAPGKE
REFVAAITWSGGQTYYQDSVKGRFTISRDNAKKLTFLQMNSLKPEDTAV
YYCAADGDRFYPEPVVDDNAYKFWGQGTQVTVSS;
or
> α7F5 (also named F5)
(SEQ ID NO: 12)
MADVQLVESGGGLVQAGGSLRLSCAASGRTFSSYSMGWFRQAPGKEREF
VAAINWSGGTTYYADSVKGRFTISRDNAKNTVYLQMNSLKPEDTAVYYC
AAGGTTAQGMSVMTPRLGSWGQGTQVTVSS.

The present invention also encompasses variant of the above VHH such as, the following VHH (CDRs are in bold and mutated amino acid compared to α7E3 underlined):

>α7E3 var1
(SEQ ID NO: 5)
MAEVQLQRSGGGRVQAGDSLRLSCAASGGTFSHYAVGWFRQAPGKEREF
VAAISWSGRSTSFANSVKGRFTISRDSAKNTAYLQMNNLKPEDTAVYCC
APARFGTGSAARDEYDDCGQGTQVTVSS
>α7E3 var2
(SEQ ID NO: 6)
MAEVQLQASGGGLVQAGDSLRLSCAASGGTFSHYAVGWFRQAPGKEREF
VAAISWSGRSTSFANSVKGRFTISRDSAKNTAYLQMRRLKPEDTAVYCC
APARFGTGSAARDEYDDCGQGTQVTVSS
>α7E3 var3
(SEQ ID NO: 7)
MAEVQLQRSGGGLVQAGDSLRLSCAASGGTFSHYAVGWFRQAPGKEREF
VAAISWSGRSTSFANSVKGRFTISRDSAKNTAYLQMRNLKPEDTAVYCC
APARFGTGSAARDEYDDCGQGTQVTVSS
>α7E3 var4
(SEQ ID NO: 8)
MAEVQLQRSGGGLVQAGDSLRLSCAASGGTFSHYAVGWFRQAPGKEREF
VAAISWSGRSTSFANSVKGRFTISRDSAKNTAYLQMRNLKPEDTAVYCC
APARFGTGSAARDEYDDCGQGTRVTVSS
>α7E3 varX
(SEQ ID NO: 9)
MAEVQLQX₁SGGGX₂VQAGDSLRLSCAASGGTFSHYAVGWFRQAPGKERE
FVAAISWSGRSTSFANSVKGRFTISRDSAKNTAYLQMX₃X₄LKPEDTAVY
CCAPARFGTGSAARDEYDDCGQGTX₅VTVSS
wherein X₁ is N or R, X₂ is A or R, X₃ is L or R,
X₄ is Q or R, X₅ is N or R.

The “MA” residues at the N terminus of the above VHH amino acid sequences are part of a cloning site which is cleaved during protein export.

The skilled person will appreciate that it may be preferable to introduce mutations in the CDRs if the VHH is to be administered. Therefore, limited mutations, which preserve the features of the antibody, more specifically single domain antibodies (for instance from camelids or sharks) and even more specifically VHH, are also included within the scope of the invention. In a particular embodiment, the CDRs of the antibody, more specifically single domain antibodies (for instance from camelids or sharks) and even more specifically VHH have limited substitutions in their amino acid sequence, preferably limited to two residues in each CDR and even more preferably to one residue. In a particular embodiment, the VHH of the invention has at least 70% identity (or more, as detailed above) with a sequence in FIG. 14 and comprises a CDR1 with a sequence having no more than 2 mismatches, preferably no more than one mismatch, with the sequence; a CDR2 with a sequence having no more than 2 mismatches, preferably no more than one mismatch, with the sequence; and a CDR3 with a sequence having no more than 2 mismatches, preferably no more than one mismatch, with the sequence.

A mismatch as meant above is preferably an amino acid substitution, in particular for CDR1 and CDR2, but may be a deletion or insertion of a single amino acid. A mismatch as meant above is preferably a conservative substitution of an amino acid, i.e. a substitution of an amino acid with another amino acid which the skilled person would realize has similar features. Portions, as defined above, of such a VHH also constitute particular embodiments. In a particular embodiment, the VHH of the invention comprises framework sequences as depicted in the sequence alignments of FIG. 14 (the framework sequences correspond to the non-bolded amino acids). In particular, the VHH of the invention may comprise the framework regions of the VHH with a sequence of FIG. 14, or with at least 80% identity and preferably at least 90% identity to the sequence of these framework regions.

The present invention also encompasses multimeric constructs. The multimeric constructs may comprise the sdAB or sdAb-comprising polypeptide of the invention, more especially the VHH or VHH-comprising polypeptide of the invention linked preferably covalently to at least a second polypeptide. The at least second polypeptide may be a single domain antibody or not.

The invention thus encompasses a VHH or VHH-comprising polypeptide as a multimeric construct, preferably a dimer construct (e.g. diabody). In one embodiment, the VHH is multivalent, preferably bivalent.

A VHH obtained from Camelidea itself usually cannot form dimers, in particular homodimers, which is thought to be one of their characteristics and required features to exert biological function. Methods to allow for the formation of dimers are known to the skilled person. In particular, such methods comprise the addition of dimerization domains, especially homodimerization domains of known dimeric proteins. Alternatively (or in addition), the presence of cysteine residues in the sequence of the VHH (e.g. by mutation of another amino acid in the sequence) or the presence of cysteines in one or more peptides bound to said VHH in the polypeptide of the invention may enable the VHH or VHH-comprising polypeptide of the invention to form dimers, especially homodimers. The skilled person will appreciate that said cysteines must be comprised in a region of the sequence such that they are accessible to binding by another polypeptide when the polypeptide is in its folded conformation. In particular embodiments, the VHH or VHH-comprising polypeptide of the invention is provided as a dimer and in particular homodimer. In particular embodiments, the VHH or VHH-comprising polypeptide of the invention comprises one or more cysteine residues which can form intermolecular disulphide bonds. In particular embodiments, the dimer-forming (especially homodimer-forming) cysteine(s) lies in the N-terminal extremity of the VHH or VHH-comprising polypeptide.

In some embodiment, at least one single domain antibody of the invention, preferably a VHH of the invention is linked to at least one other single domain antibody, preferably a VHH, directly or via one or more linker. The linker may be a peptide, peptide nucleic acid, or polyamide linkage. Suitable peptide linkers may include a plurality of amino acid residues, for example, 4, 5, 6, 7, 8, 9, 10, 15, 20 or 25 amino acids. Preferably, the linker is a GS linker such as ((Gly)₄Ser)₄ (SEQ ID NO: 55).

Each single domain antibody present in the multimeric construct may be the same.

In one embodiment, the antibody has the following amino acid sequence with E3 sequence identified in Italics, the linkers ((Gly)₄Ser)₄ (SEQ ID NO: 55) underlined:

(SEQ ID NO: 59)
EVQLQASGGGLVQAGDSLRLSCAASGGTFSHYAVGWFRQAPGKEREFVA
AISWSGRSTSFANSVKGRFTISRDSAKNTAYLQMNNLKPEDTAVYCCAP
ARFGTGSAARDEYDDCGQGTQVTVSSGGGGSGGGGSGGGGSEVQLQASG
GGLVQAGDSLRLSCAASGGTFSHYAVGWFRQAPGKEREFVAAISWSGRS
TSFANSVKGRFTISRDSAKNTAYLQMNNLKPEDTAVYCCAPARFGTGSA
ARDEYDDCGQGTQVTVSS

Alternatively, each single domain antibody present in the multimeric construct may be different than the other single domain antibodies present in the multimeric single domain antibody.

For example as disclosed below, in some embodiments, the antibody of the invention, especially the VHH or the VHH-comprising polypeptide is a fusion between the VHH targeting α7-nAChR and a VHH having a target selected from the group consisting of a membrane receptor present on the surface of the endothelial cells of the BBB, the transferrin receptor, alpha(2,3)-sialoglycoprotein receptor ((2,3)-SGPR), insulin-like growth factor 1 receptor (IGF1R); vascular cell adhesion molecule 1 (VCAM-1), prion proteins (PrPs) and transferrin receptor-1 (TfR1).

In some embodiments, the VHH or VHH-comprising polypeptide of the invention may comprise additional peptides such as tags or spacers. These comprise e.g. spacer peptides, tagging peptides and affinity peptides (and peptides having the features of both spacer peptides and tagging peptides). The VHH-comprising polypeptide of the invention may comprise one or more of such peptides. Such peptides are well known to the skilled person and only a brief description is provided herein. The skilled person will appreciate that the choice of peptide and of their position in the fusion protein should be made so that essential features of the fusion polypeptide in respect of the invention are not significantly altered. In particular, the pI should preferably remain basic, wherever possible the additional peptide should not modify the size range of the resulting polypeptide, and the addition of the peptide should not result in aggregation or sequestration (e.g. by binding to a specific cellular component) of the resulting VHH-comprising polypeptide.

A spacer peptide consists in a few amino acids which are intercalated between two defined peptides or polypeptides in a fusion protein, usually in order to allow each peptide/polypeptide to fold independently of the other, or relatively independently, i.e. in order to allow each peptide/polypeptide to adopt a conformation similar to its conformation when it is not fused to the other peptide/polypeptide. A spacer peptide may consist in a single amino acid, or a stretch of 2, 3, 4 or 5 amino acids, or 6 to 10 amino acids or 11 to 20 amino acids.

A tagging peptide, is usually used to facilitate purification and/or detection of the VHH or VHH-comprising polypeptide. In some cases, the tagging peptide is detectable by itself (e.g. fluorescent tags such as GFP) while in other cases the tagging peptide is detectable because it specifically binds a detectable molecule (in turn, the detectable molecule may be directly detectable, e.g. fluorescent, or it may be detected by specific binding to it of a detectable molecule, i.e. a scaffold of molecules may be required for detection). If used for purification and/or indirect detection, such a peptide is usually designed (or found) to have a high affinity to a readily available molecule. Such peptides are often derived from a species unrelated to the species where the polypeptides is intended to be used to avoid any cross reaction, especially during detection. The molecule binding the tagging peptide may be selected for its detectability and/or for ease of immobilization and/or recovery in purification processes. Common tagging peptide include HA-tag (a short peptide from human influenza hemagglutinin), Flag-tag, His-tag (comprising at least 6 histidine residues) and the Strep-tag (comprising eight amino acids and which is readily bound by commercially available Streptavidin and antibodies). In particular embodiments, the VHH-comprising polypeptide of the invention comprises a Strep-tag, in particular fused C-terminally to the VHH.

In an embodiment, the multimeric construct is or comprises a fusion protein comprising an antibody of the invention, especially a sdAB or sdAb-comprising polypeptide of the invention more especially the VHH or VHH-comprising polypeptide of the invention covalently linked to at least a second polypeptide that is not a single domain antibody.

As used herein a “fusion protein” refers to a polypeptide having two or more portions covalently linked together, where each of the portions is a polypeptide having preferably at least one different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the at least two portions and the linker will be in reading frame with each other. Preferably, the at least two portions of the polypeptide are obtained from heterologous or different polypeptides. In the context of this invention, one of the portions is a single domain antibody, preferably a VHH of the invention.

In the fusion protein of the present invention, the antibody of the invention may be directly fused or linked via a linker moiety to the other elements of the fusion protein.

The linker may be for example AAARSDKTHTCPPCPAPELLG (SEQ ID NO: 54). Also included is a sequence, which has at least 50%, 60%, 70%, 80%, 90%, 95% or 99% identity thereto, using the default parameters of the BLAST computer program provided by HGMP.

Other examples of linkers are given in Xiaoying Chen et al. Fusion protein linkers: property, design and functionality, Adv Drug Deliv Rev. 2013 October; 65 (10): 1357-69.

In some embodiments of the fusion protein, the second polypeptide is selected from a Fab, Fc, F(ab′)2 (including chemically linked F(ab′)2 chains), Fab′, scFv (including multimer forms thereof, i.e. di-scFv, or tri-scFv), or BiTE (bi-specific T-cell engager).

In some embodiments the second polypeptide is a Fc fragment of a mammalian immunoglobulin. In some embodiments the mammal is a human. In some embodiments, the Fc fragment is a Fc fragment from IgG1 and has the amino acid sequence of SEQ ID NO: 48.

(SEQ ID NO: 48)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEAL
HNHYTQKSLSLSPGK

Advantageously, the Fc portion of the fusion proteins comprising Fc fragment is responsible for linking these fusion proteins together into a homodimeric form. This homodimeric form comprises two fusion proteins linked together through their Fc portion.

Thus, when the fusion protein comprising Fc fragment is expressed in a suitable recombinant cell type, respectively homodimer of the fusion proteins are formed.

Therefore, the present invention encompasses both the fusion proteins comprising a Fc fragment in their monomeric and in their homodimeric forms.

Moreover, the fusion of the antibody of the invention, especially a sdAB or sdAb-comprising polypeptide of the invention more especially the VHH or VHH-comprising polypeptide of the invention with a Fc fragment enhances its affinity for alpha7 nAChR.

In some embodiments, the VHH or VHH-comprising polypeptide is a fusion with human immunoglobulin fragment crystallizable region (Fc).

In various embodiments, the VHH has the following amino acid sequence:

> α7E3-Fc (with E3 sequence in Italics and Fc
sequence (SEQ ID NO: 48) in Bold:
(SEQ ID NO: 45)
MGSSHHHHHHAAAAEVQLQASGGGLVQAGDSLRLSCAASGGTFSHYAV
GWFRQAPGKEREFVAAISWSGRSTSFANSVKGRFTISRDSAKNTAYLQ
MNNLKPEDTAVYCCAPARFGTGSAARDEYDDCGQGTQVTVSSAAARSD
KTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSL
TCLVKGFYPSDIAVEWESNGQPENNYKTTPPVLDSDGSFFLYSKLTVD
KSRWQQGNVFSCSVMHEALHNHYTQKSLSLSPGK;
or
> α7C4-Fc (with C4 sequence in Italics, linker
sequence (SEQ ID NO: 54) underlined and
Fc sequence (SEQ ID NO: 48) in Bold:
(SEQ ID NO: 46)
MGSSHHHHHHAAAAQVQLVESGGGLVQAGGSLKLSCAASGFTFAHYAM
VWFRQAPGKEREFVAGISWSGASTYYASSVKGRFTISRDNAKNTVYLQ
MNSLKPEDTAVYYVAAARFGVGVDDDYSYWGQGTQVTVSSAAARSDKT
HTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTC
LVKGFYPSDIAVEWESNGQPENNYKTTPPVLDSDGSFFLYSKLTVDKS
RWQQGNVFSCSVMHEALHNHYTQKSLSLSPGK.

In another embodiment, the antibody, more specifically single domain antibodies (for instance from camelids or sharks) and even more specifically VHH is engineered to cross the blood-brain barrier (BBB) and/or to increase its ability to cross the BBB. The ability of said antibodies to cross the BBB can be assayed by studying the passage on microfluidic devices containing a semi-permeable membrane where confluent mice brain endothelial cells are grown. The VHH of the invention can be engineered to cross the BBB by introduction of positive charges in the VHH, for example, as detailed in U.S. Pat. No. 9,387,260, which is incorporated by reference herein, where it is shown that VHH that have an intracellular target in a brain cell and an isoelectric point above 8.5 can cross the BBB.

In some embodiments, the VHH, especially α7E3, has one or more of the following mutations:
Alanine (A) in position 20 can be mutated in Arginine (R)
Leucine (L) in position 25 can be mutated in Arginine (R)
Asparagine (N) in position 98 can be mutated in Arginine (R)
Asparagine (N) in position 99 can be mutated in Arginine (R)
Glutamic acid (Q) in position 133 can be mutated in Arginine (R)

In some embodiments, the VHH has one or more of the following α7E3 variant amino acid sequences, showing an isoelectric point above 8.5:

Variants comprising at least one of the above
mutations are disclosed below. The His-tag (SEQ
ID NO: 58) and CSA linker (SEQ ID NO: 56) are
underlined.
> α7E3CSA var1
(SEQ ID NO: 40)
MGSSHHHHHHAAAAEVQLQRSGGGRVQAGDSLRLSCAASGGTFSHYA
VGWFRQAPGKEREFVAAISWSGRSTSFANSVKGRFTISRDSAKNTAY
LQMNNLKPEDTAVYCCAPARFGTGSAARDEYDDCGQGTQVTVSSGGG
GSGGGGSGGGGSCSA
> α7E3CSA var2
(SEQ ID NO: 41)
MGSSHHHHHHAAAAEVQLQASGGGLVQAGDSLRLSCAASGGTFSHYA
VGWFRQAPGKEREFVAAISWSGRSTSFANSVKGRFTISRDSAKNTAY
LQMRRLKPEDTAVYCCAPARFGTGSAARDEYDDCGQGTQVTVSSGGG
GSGGGGSGGGGSCSA
> α7E3CSA var3
(SEQ ID NO: 42)
MGSSHHHHHHAAAAEVQLQRSGGGLVQAGDSLRLSCAASGGTFSHYA
VGWFRQAPGKEREFVAAISWSGRSTSFANSVKGRFTISRDSAKNTAY
LQMRNLKPEDTAVYCCAPARFGTGSAARDEYDDCGQGTQVTVSSGGG
GSGGGGSGGGGSCSA
> α7E3CSA var4
(SEQ ID NO: 43)
MGSSHHHHHHAAAAEVQLQRSGGGLVQAGDSLRLSCAASGGTFSHYA
VGWFRQAPGKEREFVAAISWSGRSTSFANSVKGRFTISRDSAKNTAY
LQMRNLKPEDTAVYCCAPARFGTGSAARDEYDDCGQGTRVTVSSGGG
GSGGGGSGGGGSCSA.

In some embodiments, the antibody of the invention, especially the VHH or the VHH-comprising polypeptide is a fusion between the VHH targeting α7-nAChR and a VHH targeting a membrane receptor present on the surface of the endothelial cells of the BBB. In some embodiments, the VHH is a fusion between the VHH targeting α7-nAChR and a VHH targeting the transferrin receptor, alpha(2,3)-sialoglycoprotein receptor ((2,3)-SGPR), insulin-like growth factor 1 receptor (IGF1R); vascular cell adhesion molecule 1 (VCAM-1), or prion proteins (PrPs), transferrin receptor-1 (TfR1).

In certain embodiments the at least 2 VHHs may be linked together via one or more type of linker. The linker may be a peptide, peptide nucleic acid, or polyamide linkage. Suitable peptide linkers may include a plurality of amino acid residues, for example, 4, 5, 6, 7, 8, 9, 10, 15, 20 or 25 amino acids. Preferably, the linker is a GS linker such as ((Gly)₄Ser)₄ (SEQ ID NO: 55).

In one embodiment, the VHH has the following amino acid sequence:

>α7E3E3-TfR-CSA (with E3 sequence identified in
Italics, the linkers ((Gly)₄Ser)₄ (SEQ ID NO: 55)
underlined and TfR sequence (SEQ ID NO: 47)
identified in Bold):
(SEQ ID NO: 44)
MGSSHHHHHHSSGLVPRGSAAAEVQLQASGGGLVQAGDSLRLSCAASG
GTFSHYAVGWFRQAPGKEREFVAAISWSGRSTSFANSVKGRFTISRDS
AKNTAYLQMNNLKPEDTAVYCCAPARFGTGSAARDEYDDCGQGTQVTV
SSGGGGSGGGGGGGGSEVQLQASGGGLVQAGDSLRLSCAASGGTFSHY
AVGWFRQAPGKEREFVAAISWSGRSTSFANSVKGRFTISRDSAKNTAY
LQMNNLKPEDTAVYCCAPARFGTGSAARDEYDDCGQGTQVTVSSGGGG
SGGGGSGGGGSQVQLQESGPGLVKPSETLSLTCTVSAGSISSTSTSYY
WGWIRQSPGKGLEWIGSIYYSGRTYYNPSLKSRVTISVDRPNNQFSLK
VSSVTAADSAVYYCARHRRVLLWIGELLDDYDRDVWGQGTMVTVSSGG
GGSGGGGSGGGGSCSA.

α7E3E3-TfR-CSA was deposited on Nov. 8, 2022 at the Collection Nationale de Cultures des Microorganismes, 25 rue du Docteur Roux, 75015 Paris, France under No. CNCM 1-5917.

In one embodiment the antibody is E3TfrA2 and has the following sequence (with E3 sequence identified in Italics, the linker underlined and VHH directed to the transferrin receptor TfRA2 sequence identified in Bold):

(SEQ ID NO: 70)
MAEVQLQASGGGLVQAGDSLRLSCAASGGTFSHYAVGWFRQAPGKEREF
VAAISWSGRSTSFANSVKGRFTISRDSAKNTAYLQMNNLKPEDTAVYCC
APARFGTGSAARDEYDDCGQGTQVTVSSGGGGSGGGGSGGGGSEVQLVE
SGGGVVQPGGSLKLSCVASGTDFSINFIRWYRQAPGKQREFVAGFTATG
NTNYADSMKGRFTISRDNTKNAVYLQIDSLKPEDTAVYYCYMLDKWGQG
THVTVSSGGGGSHHHHHHGSCSA

In one embodiment the antibody is E3TfrB2 and has the following sequence (with E3 sequence identified in Italics, the linker underlined and VHH directed to the transferrin receptor TfRB2 sequence identified in Bold):

(SEQ ID NO: 71)
MAEVQLQASGGGLVQAGDSLRLSCAASGGTFSHYAVGWFRQAPGKEREF
VAAISWSGRSTSFANSVKGRFTISRDSAKNTAYLQMNNLKPEDTAVYCC
APARFGTGSAARDEYDDCGQGTQVTVSSGGGGSGGGGSGGGGSEVQLVE
SGGGVVQPGGSLRLSCAASGEIFSINFMRWYRQAPGKQREWVAGFTRDG
STNYPDSAKGRFTISRDNAKNTVYLQIDSLKPEDTAVYYCYMLDTWGQG
TQVTVSSGGGGSHHHHHHGSCSA

### C. Nucleic Acids Encoding Antibodies

The invention encompasses DNAs and RNAs encoding any of the antibodies of the invention. In one embodiment, the VHH can be encoded by any of the following DNA sequences:

> α7E3
(SEQ ID NO: 49)
ATGGCCGAGGTCCAGCTGCAGGCGTCTGGGGGAGGATTGGTGCAGGCTGGGGAC
TCTCTGAGACTCTCTTGTGCAGCCTCTGGAGGCACCTTCAGTCACTATGCCGTGGGCTGGT
TCCGCCAGGCTCCAGGGAAGGAGCGTGAGTTCGTAGCAGCTATTAGCTGGAGTGGTCGTA
GCACAAGCTTTGCAAACTCCGTGAAGGGCCGATTCACCATCTCCAGAGACAGCGCCAAGA
ATACGGCCTATCTACAAATGAACAACCTGAAACCTGAGGACACGGCCGTTTATTGTTGTGC
ACCAGCCCGATTTGGTACTGGATCAGCGGCGCGGGATGAGTATGACGACTGCGGCCAGG
GGACCCAGGTCACCGTCTCCTCAGCGGCCGCAGAACAAAAACTCATCTCAGAAGAGGATC
TGAATGGGGCCGCACATCACCACCATCACCATGGGAGC
> α7C4
(SEQ ID NO: 50)
ATGGCTCAGGTGCAGCTCGTGGAGTCCGGGGGAGGATTGGTGCAGGCTGGGGGC
TCTCTGAAACTCTCCTGTGCAGCCTCTGGATTCACCTTCGCTCACTATGCCATGGTCTGGTT
CCGCCAGGCTCCAGGGAAGGAGCGTGAGTTTGTAGCAGGCATTAGCTGGAGTGGTGCTAG
CACATACTATGCAAGCTCCGTGAAGGGCCGATTCACCATCTCCAGAGACAACGCCAAGAAC
ACGGTGTATCTGCAAATGAACAGCCTGAAACCTGAGGACACGGCCGTTTATTACGTTGCAG
CGGCTAGGTTTGGGGTTGGGGTCGACGATGACTATTCCTACTGGGGCCAGGGGACCCAG
GTCACCGTCTCCTCAGCGCACCACAGCGAAGACCCCAGCGCGGCCGCAGAACAAAAACTC
ATCTCAGAAGAGGATCTGAATGGGGCCGCACATCACCACCATCACCATGGGAGC
> α7E3E3TfR
(SEQ ID NO: 51)
CCATGGGCAGCAGCCATCATCATCATCATCACAGCAGCGGCCTGGTGCCGCGCGG
CAGCGCTGCTGCCGAGGTCCAGCTGCAGGCGTCTGGGGGAGGATTGGTGCAGGCTGGGG
ACTCTCTGAGACTCTCTTGTGCAGCCTCTGGAGGCACCTTCAGTCACTATGCCGTGGGCTG
GTTCCGCCAGGCTCCAGGGAAGGAGCGTGAGTTCGTAGCAGCTATTAGCTGGAGTGGTCG
TAGCACAAGCTTTGCAAACTCCGTGAAGGGCCGATTCACCATCTCCAGAGACAGCGCCAAG
AATACGGCCTATCTACAAATGAACAACCTGAAACCTGAGGACACGGCCGTTTATTGTTGTG
CACCAGCCCGATTTGGTACTGGATCAGCGGCGCGGGATGAGTATGACGACTGCGGCCAG
GGGACCCAAGTCACCGTCTCCTCAGGGGGCGGAGGTAGTGGTGGAGGTGGAAGTGGAGG
TGGGGGGTCTGGTGGGGGGGGTCCGAAGTTCAATTGCAGGCTAGTGGCGGCGGTTTAG
TGCAGGCCGGTGACTCTCTCCGCCTGTCCTGCGCGGCATCGGGCGGCACATTCAGCCATT
ATGCCGTGGGCTGGTTTCGCCAGGCACCGGGCAAGGAGCGTGAATTCGTCGCTGCCATTT
CGTGGAGTGGCCGGTCGACTAGCTTTGCAAACAGCGTTAAAGGCCGATTTACCATCTCACG
CGACTCCGCCAAAAACACGGCGTACCTGCAGATGAATAATCTGAAACCAGAGGATACGGC
GGTATATTGCTGTGCTCCGGCGCGTTTCGGTACCGGTAGCGCGGCGCGTGATGAATACGA
TGATTGTGGCCAGGGTACCCAGGTCACAGTGTCAAGCGGCGGCGGTGGAAGTGGGGGG
GAGGCAGCGGTGGAGGTGGATCCCAGGTGCAGCTGCAGGAGAGCGGCCCCGGCCTGGT
GAAGCCCAGCGAGACCCTGAGCCTGACCTGCACCGTGAGCGCCGGCAGCATCAGCAGCA
CCAGCACCAGCTACTACTGGGGCTGGATCAGGCAGAGCCCCGGCAAGGGCCTGGAGTGG
ATCGGCAGCATCTACTACAGCGGCAGGACCTACTACAACCCCAGCCTGAAGAGCAGGGTG
ACTATCAGCGTGGACAGGCCCAACAACCAGTTCAGCCTGAAGGTGAGCAGCGTGACCGCC
GCCGACAGCGCCGTGTACTACTGCGCCAGGCACAGGAGGGTGCTGCTGTGGATCGGCGA
GCTGCTGAACAACTACAACAGGAACGTGTGGGGCCAGGGCACAATGGTGACCGTGAGCAG
CGGTGGTGGAGGCAGCGGGGGTGGAGGTAGCGGCGGAGGTGGTAGCTGTAGCGCTTAGT
AAGCGGCCGCGTGCTAGC
> α7E3-Fc
(SEQ ID NO: 52)
ATGGGCAGCAGCCATCATCATCATCATCACGCTGCTGCTGCCGAGGTCCAGCTGCA
GGCGTCTGGGGGAGGATTGGTGCAGGCTGGGGACTCTCTGAGACTCTCTTGTGCAGCCTC
TGGAGGCACCTTCAGTCACTATGCCGTGGGCTGGTTCCGCCAGGCTCCAGGGAAGGAGC
GTGAGTTCGTAGCAGCTATTAGCTGGAGTGGTCGTAGCACAAGCTTTGCAAACTCCGTGAA
GGGCCGATTCACCATCTCCAGAGACAGCGCCAAGAATACGGCCTATCTACAAATGAACAAC
CTGAAACCTGAGGACACGGCCGTTTATTGTTGTGCACCAGCCCGATTTGGTACTGGATCAG
CGGCGCGGGATGAGTATGACGACTGCGGCCAGGGGACCCAGGTCACCGTGTCTAGCGCG
GCCGCTAGATCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCT
CCAAAGCCAAAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACAAGAGCAG
GTGGCAGCAGGGGAACGTCTTCTCATGCTCCGTGATGCACGAGGCTCTGCACAACCACTA
CACGCAGAAGAGCCTCTCCCTGTCTCCGGGTAAATGA
> α7C4-Fc
(SEQ ID NO: 53)
ATGGGCAGCAGCCATCATCATCATCATCACGCTGCTGCTGCTCAGGTGCAGCTCGT
GGAGTCCGGGGGAGGATTGGTGCAGGCTGGGGGCTCTCTGAAACTCTCCTGTGCAGCCT
CTGGATTCACCTTCGCTCACTATGCTATGGTCTGGTTCCGCCAGGCTCCAGGGAAGGAGC
GTGAGTTTGTAGCAGGCATTAGCTGGAGTGGTGCTAGCACATACTATGCAAGCTCCGTGAA
GGGCCGATTCACCATCTCCAGAGACAACGCCAAGAACACGGTGTATCTGCAAATGAACAGC
CTGAAACCTGAGGACACGGCCGTTTATTACGTTGCAGCGGCTCGTTTTGGGGTTGGGGTC
GACGATGACTATTCCTACTGGGGCCAGGGGACCCAGGTCACCGTGTCTAGCGCGGCCGCT
AGATCTGACAAAACTCACACATGCCCACCGTGCCCAGCACCTGAACTCCTGGGGGGACCG
TCAGTCTTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATG
ACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCT
GGACTCCGACGGCTCCTTCTTCCTCTACAGCAAGCTCACCGTGGACAAGAGCAGGTGGCA
GCAGGGGAACGTCTTCTCATGCTCCGTGATGCACGAGGCTCTGCACAACCACTACACGCA
GAAGAGCCTCTCCCTGTCTCCGGGTAAATGA

In one embodiment, the nucleic acid encodes an VHH with at least 50%, 60%, 70%, 80%, 90%, 93%, 95%, 97%, 98%, 99% or 100% identity with a VHH of the invention. In one embodiment, the nucleic acid encodes an VHH with 50%, 60%, 70%, 80%, 90%, 93%, 95%, 97%, 98%, 99% or 100% identity with any of the VHH sequences in FIG. 14. Due to degeneracy of the genetic code, many different DNAs can code for the antibodies of the invention.

The invention encompasses a recombinant vector comprising a nucleic acid encoding a VHH of the invention. Vectors, include plasmids, expression vectors, cosmids, phages, phagemids, viruses, etc.

The invention encompasses a recombinant vector for expression of a VHH of the invention. The recombinant vector can be a vector for eukaryotic or prokaryotic expression, such as a plasmid, a phage for bacterium introduction, a YAC able to transform yeast, a viral vector and especially a retroviral vector, or any expression vector. An expression vector as defined herein is chosen to enable the production of a VHH of the invention, either in vitro or in vivo.

In one embodiment, the expression vector encodes a VHH with at least 50%, 60%, 70%, 80%, 90%, 93%, 95%, 97%, 98%, 99% or 100% identity with a VHH of the invention. In one embodiment, the expression vector encodes a VHH with 50%, 60%, 70%, 80%, 90%, 93%, 95%, 97%, 98%, 99% or 100% identity with any of the VHH sequences in FIG. 14.

In one embodiment, the expression vector encodes a protein purification tag. In one embodiment, the expression vector encodes a protease cleavage site, such as TEV or Thrombin cleavage site, inserted between the VHH coding sequence and a protein purification tag, such as polyHis tag. In a preferred embodiment, the expression vector encodes a His tag. In one embodiment, a protease cleavage site is positioned to remove the His tag, for example, after purification.

The expression vector can comprise transcription regulation regions (including promoter, enhancer, ribosome binding site (RBS), polyA signal), a termination signal, a prokaryotic or eukaryotic origin of replication and/or a selection gene. The features of the promoter can be easily determined by the man skilled in the art in view of the expression needed, i.e., constitutive, transitory or inducible (e.g. IPTG), strong or weak, tissue-specific and/or developmental stage-specific promoter. The vector can also comprise sequence enabling conditional expression, such as sequences of the Cre/Lox system or analogue systems.

The nucleic acid molecules according to the invention can be obtained by known conventional methods, following standard protocols such as those described in Current Protocols in Molecular Biology (Frederick M. AUSUBEL, 2000, Wiley and son Inc., Library of Congress, USA). For example, they may be obtained by amplification of a nucleic sequence by PCR or RT-PCR or alternatively by total or partial chemical synthesis.

The vectors are constructed and introduced into host cells by conventional recombinant DNA and genetic engineering methods which are known per se. Numerous vectors into which a nucleic acid molecule of interest may be inserted in order to introduce it and to maintain it in a host cell are known per se; the choice of an appropriate vector depends on the use envisaged for this vector (for example replication of the sequence of interest, expression of this sequence, maintenance of the sequence in extrachromosomal form or alternatively integration into the chromosomal material of the host), and on the nature of the host cell.

The invention also provides vectors such as plasmids or viruses containing one or more of the nucleic acid molecules of the invention. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989). Any of a variety of expression vectors known to those of ordinary skill in the art can be employed to express recombinant polypeptides of this invention. Expression can be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast, insect cells, or a mammalian cell line such as COS or CHO. The DNA sequences expressed in this manner can encode any of the polypeptides described herein including variants thereof.

### D. Cells

The invention encompasses cells comprising the nucleic acids and vectors of the invention. The cells can be eukaryotic (e.g., yeast or mammalian) or prokaryotic (e.g., bacterial) cells. Preferably, the cells are mammalian cells, preferably cultured cells. In some embodiments, the cells are a mammalian cell line suitable for growth in a suspension. In some embodiments, the cells are a transformed cell line. Preferably, the cells constitutively produce a VHH of the invention. In some embodiments, the production of a VHH of the invention is inducible.

The invention also encompasses a method of preparing an antibody, more specifically single domain antibodies (for instance from camelids or sharks) and even more specifically a VHH comprising culturing cells comprising an expression vector of the invention and recovering the expressed antibody, preferably VHH. The invention further encompasses the antibody, more specifically single domain antibodies (for instance from camelids or sharks) and even more specifically VHH produced by these methods from the nucleic acids of the invention.

Uses of vectors such as plasmids or viruses containing the nucleic acids of the present invention include generation of mRNA or protein in vitro or in vivo. In related embodiments, the methods, compositions and kits encompass host cells transformed with the vectors described above. Nucleic acid molecules can be inserted into a construct which can, optionally, replicate and/or integrate into a recombinant host cell, by known methods. The host cell can be a eukaryote or prokaryote and includes, for example, yeast (such as Pichia pastoris or Saccharomyces cerevisiae), bacteria (such as E. coli, or Bacillus subtilis), animal cells or tissue, insect Sf9 cells (such as baculoviruses infected SF9 cells) or mammalian cells (somatic or embryonic cells, Human Embryonic Kidney (HEK and EXPI293F) cells, Chinese hamster ovary cells, Hela cells, human 293 cells and monkey COS-7 cells). Host cells suitable in the present invention also include a mammalian cell and a plant cell.

The nucleic acid molecule can be incorporated or inserted into the host cell by known methods. Examples of suitable methods of transfecting or transforming cells include calcium phosphate precipitation, electroporation, microinjection, infection, lipofection and direct uptake. “Transformation” or “transfection” as used herein refers to the acquisition of new or altered genetic features by incorporation of additional nucleic acids, e.g., DNA. “Expression” of the genetic information of a host cell is a term of art which refers to the directed transcription of DNA to generate RNA which is translated into a polypeptide. Methods for preparing such recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), for example.

The host cell is maintained under suitable conditions for expression and recovery of the polypeptides of the present invention. In certain embodiments, the cells are maintained in a suitable buffer and/or growth medium or nutrient source for growth of the cells and expression of the gene product(s). The growth media are not critical to the invention, are generally known in the art and include sources of carbon, nitrogen and sulfur. Examples include Luria-Bertani broth, Superbroth, Dulbecco's Modified Eagles Media (DMEM), RPMI-1640, M199 and Grace's insect media. The growth media can contain a buffer, the selection of which is not critical to the invention. The pH of the buffered Media can be selected and is generally one tolerated by or optimal for growth for the host cell.

The host cell is maintained under a suitable temperature and other suitable conditions for growth. The temperature is selected so that the host cell tolerates the process and is for example, between about 13-40° Celsius.

### E. Treatment of Cognitive Disorders

The invention encompasses compositions comprising an antibody of the invention for treatment, preferably for treatment of cognitive disorders, uses of these compositions for treatment, preferably for treatment of cognitive disorders, and methods of treatment, preferably for treatment of cognitive disorders with these compositions, uses of these compositions in the manufacture of a medicament, preferably a medicament for treating cognitive disorder.

In an embodiment of the invention, the composition comprising an antibody of the invention is for use in a method of treatment, preferably a treatment of cognitive disorders.

In one embodiment, the method comprises administering an antibody of the invention to a patient. The patient is a patient in need thereof. For example, the patient is a patient having cognitive disorders such as object recognition, social recognition, working memory, executive function, spatial reference memory, recognition memory, and spatial learning. The patient may also be a patient suffering of a disease selected from the group consisting of Alzheimer's disease, Parkinson's disease and schizophrenia.

The antibody comprised in the composition may be any antibody according to the section B.

In a preferred embodiment of the invention, the composition for use in a method of treatment comprises the VHH comprising or consisting of VHH α7E3 (SEQ ID NO: 4) or a variant having at least 70% or at least 80% identity, at least 90% identity, at least 95% identity or at least 99% identity thereof, preferably a variant having an amino acid selected from the group consisting of SEQ ID NO:5, SEQ ID NO: 6, SEQ ID NO: 7, SEQ ID NO: 8 and SEQ ID NO: 9.

Examples of cognitive disorders that can be treated are object recognition, social recognition, working memory, executive function, spatial reference memory, recognition memory, and spatial learning.

In an embodiment, the composition according to the invention is for use in a method of treatment of a disease selected from the group consisting of Alzheimer's disease, Parkinson's disease and schizophrenia.

In a particular embodiment of the invention, the composition further comprises a pharmaceutically acceptable vehicle. As defined herein, a pharmaceutically acceptable vehicle encompasses any substance that enables the formulation of the antibody, preferably the VHH, the polynucleotide, or the vector according to the invention within a composition. A vehicle is any substance or combination of substances physiologically acceptable i.e., appropriate for its use in a composition in contact with a host, especially a human, and thus non-toxic. Examples of such vehicles are phosphate buffered saline solutions, distilled water, emulsions such as oil/water emulsions, various types of wetting agents sterile solutions and the like.

In another particular embodiment of the invention, the composition is formulated for an administration through parenteral route such as subcutaneous (s.c.), intradermal (i.d.), intramuscular (i.m.), intraperitoneal (i.p.) or intravenous (i.v.) injection.

In another particular embodiment of the invention, the composition is administered in one or multiple administration dose(s).

The quantity to be administered (dosage) depends on the subject to be treated, including the condition of the patient, the state of the individual's immune system, the route of administration and the size of the host. Suitable dosage ranges can be determined by the skilled artisan and can be modified by one skilled in the art, depending on circumstances.

In a preferred embodiment of the invention, the composition is for use for treatment of cognitive disorders.

Methods of Detection of α7 nAChR
In another aspect, methods for detection of α7 nAChR are provided.
The methods may comprise providing a detection agent comprising an anti-α7 nAChR antibody according to the invention; providing a biological sample; contacting the detection agent with the biological sample; and visualizing the antigen-detection agent complexes formed.
In an embodiment, the detection agent further comprises a label.
Preferred labels include a fluorescent label, such as FITC or AlexaFluor647NHS, a chromophore label, an affinity-ligand label, an enzyme label, such as alkaline phosphatase or a luciferase, horseradish peroxidase, or β galactosidase, an enzyme cofactor label, a hapten conjugate label, such as digoxigenin or dinitrophenyl, a Raman signal generating label, a magnetic label, a spin label, an epitope label, such as the FLAG or HA epitope, a luminescent label, a heavy atom label, a nanoparticle label, an electrochemical label, a light scattering label, a spherical shell label, semiconductor nanocrystal label, wherein the label can allow visualization with or without a secondary detection molecule.
Preferred labels include suitable enzymes such as horseradish peroxidase, alkaline phosphatase, beta-galactosidase, luciferase or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, Alexa fluorescent protein, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, as well as others known in the art; a luminescent material such as luminol; light scattering or plasmon resonant materials such as gold or silver particles or quantum dots; or radioactive material include ⁴C, ¹²³I, ¹²⁴I, ¹²⁵I, ³²P, ³³P, ³⁵S, or ³H.
More preferably, the label is selected from a chemiluminescent label, an enzyme label, a fluorescence label, and a radioactive (e.g., iodine) label.
In an embodiment, the step of visualizing the antigen-detection agent complexes formed comprises visualizing the label.
In one embodiment, the detection agent is attached to an appropriate support, in particular a microplate or a bead.
The detection agent according to the invention is useful for the direct detection of α7 nAChR; the detection of α7 nAChR can be carried out by an appropriate technique, in particular EIA, ELISA, RIA, immunofluorescence, luminescence in a biological sample.
Therefore, the present invention encompasses the use of the detection agent for the detection of α7 nAChR.
A skilled artisan will appreciate that the detection agent can be used in any suitable assay format known in the art that is designed to utilize antibodies.
In some embodiments, the detection agent comprises the anti-α7 nAChR antibody selected from the VHHs having the amino acid sequence selected from the group consisting of SEQ ID NO: 1 to SEQ ID NO: 12, the fusion protein comprising any one of these VHHs fused to a Fc fragment such as α7E3-Fc (SEQ ID NO: 45) or α7C4-Fc (SEQ ID NO: 46), the fusion protein comprising any one of these VHHs fused to CSA such as α7E3CSA var1 (SEQ ID NO: 40), α7E3CSA var2 (SEQ ID NO: 41), α7E3CSA var3 (SEQ ID NO: 42), or α7E3CSA var4 (SEQ ID NO: 43), and variant thereof having at least 70% or at least 80% identity, at least 90% identity, at least 95% identity or at least 99% identity thereof. In a preferred embodiment, the detection agent comprises the VHH C4 having SEQ ID NO: 3, the fusion protein comprising VHH C4 fused to a Fc fragment, the fusion protein comprising VHH C4 fused to CSA and/or variant having at least 70% or at least 80% identity, at least 90% identity, at least 95% identity or at least 99% identity thereof.
The invention encompasses a composition comprising a detection agent comprising an anti-α7 nAChR antibody for use in the detection of α7 nAChR, in a biological sample.
Preferably, the method comprises comparing the results obtained with a positive and/or negative control.
In various embodiments, the method comprises quantification of the α7 nAChR detected.

### EXAMPLES

### Example 1-7: Results

### Example 1. Isolation of VHHs from Alpacas Immunized with Cells Expressing the α7-5-HT₃A Chimera

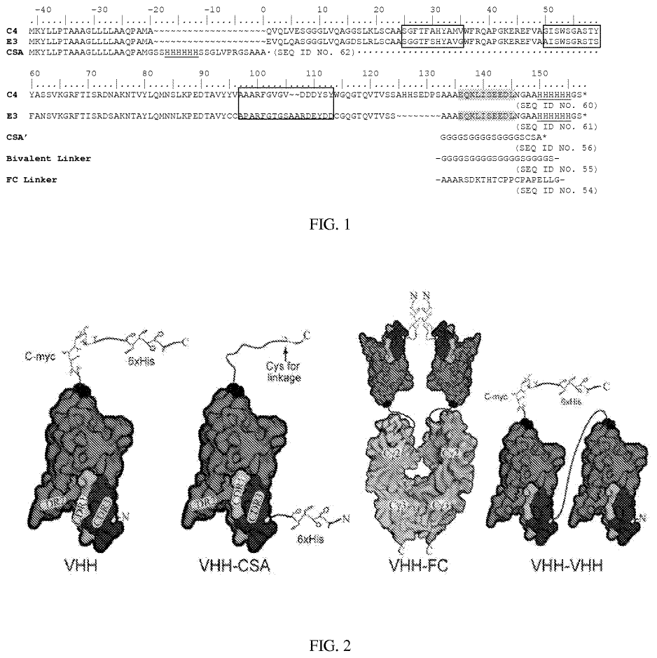

To avoid the time-consuming and expensive production and purification of the α7 receptor, alpacas were immunized with HEK 293 cells transiently transfected with a cDNA directing the expression of an α7/5-HT₃A chimera. This chimera, where the ECD of the human α7-nAChR is fused to the TMD of the mouse 5-HT₃A receptor, is expressed at the surface of the cells at much higher levels than α7-nAChR (Craig et al., 2004) (Corringer et al., 1995). In addition, injecting transfected cells ensures that the receptor is correctly folded and inserted in the membrane as compared to detergent solubilized receptor, increasing the chances to isolate conformation-specific VHHs. A similar approach has been successful for other membrane proteins such as the metabotropic glutamate receptors (Scholler et al., 2017). After immunization, the VHH encoding sequences were amplified from the serum and a phage display library was generated. The library was depleted by several rounds of incubation with non-transfected HEK 293 and VERO cells, after which VERO cells transfected with the α7/5-HT₃A chimera were used to select phages binding to the α7 ECD in a single round of panning. Isolated clones from the final two rounds of panning were then screened by an ELISA against transfected VERO cells. Seven unique sequences have been obtained from the second and the third rounds of panning. (FIG. 1; FIG. 14).

### Example 2. Immunofluorescence Experiments Show that α7C4 and α7E3 are Specific for the α7-nAChR Over α3β4- and α4β2-nAChRs

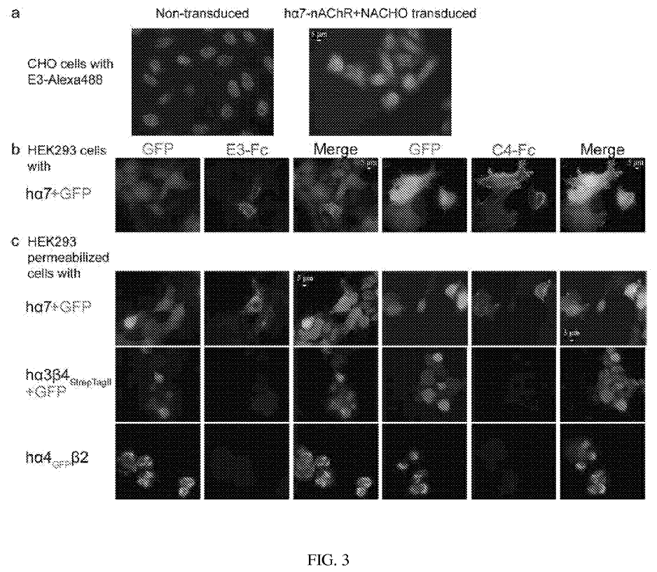

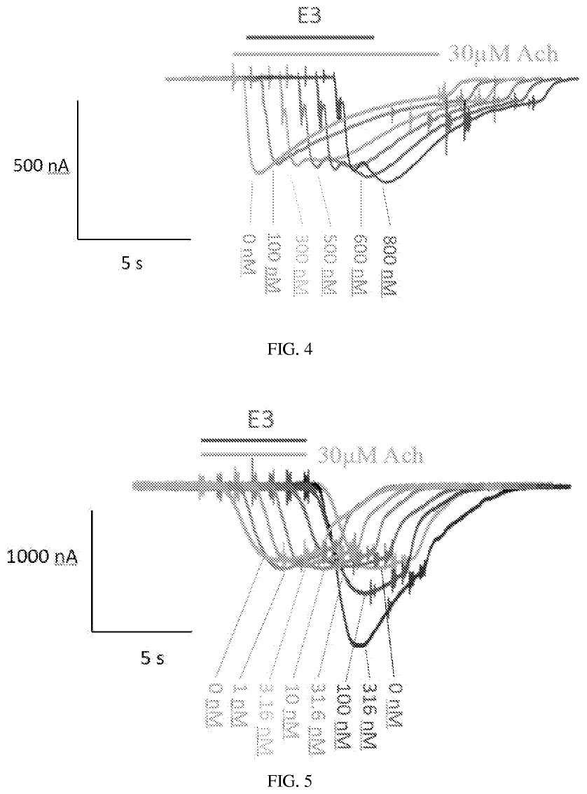

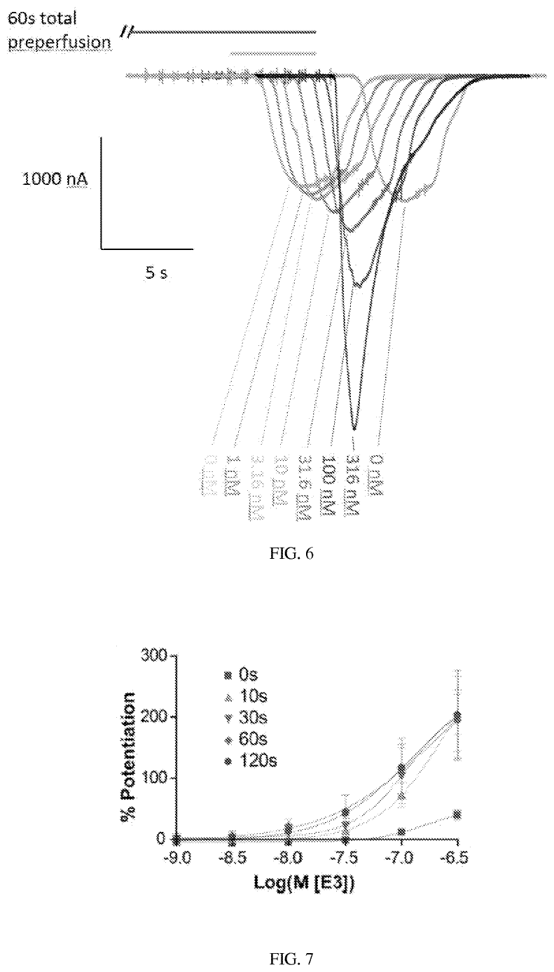

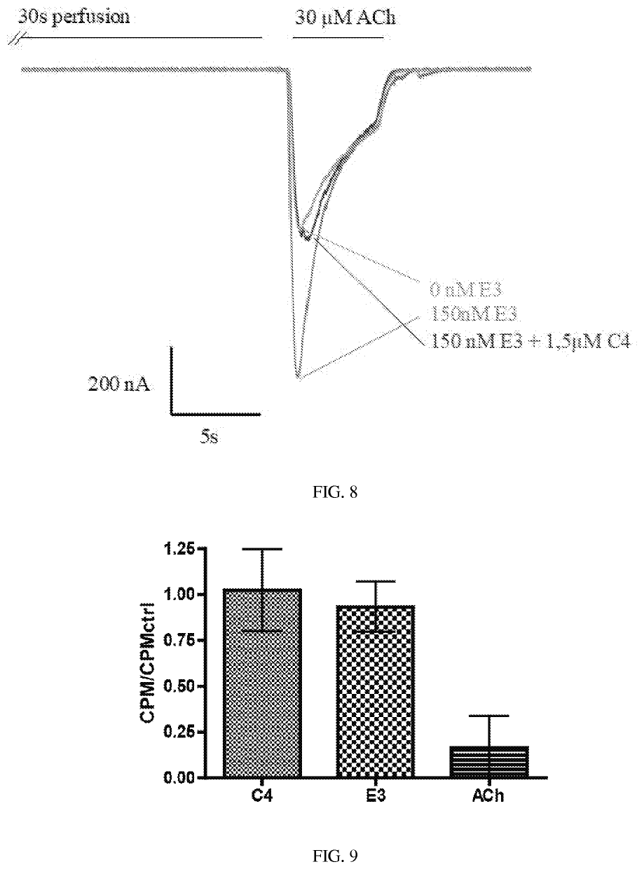

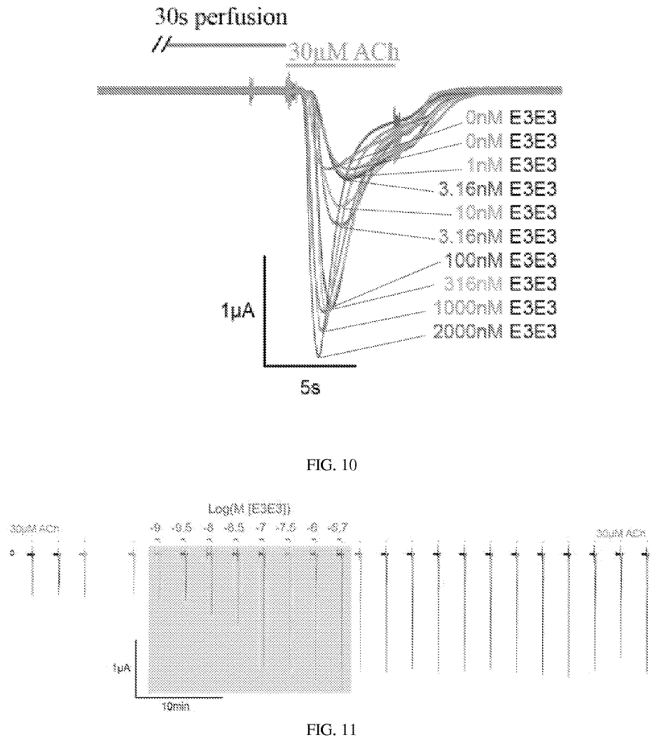

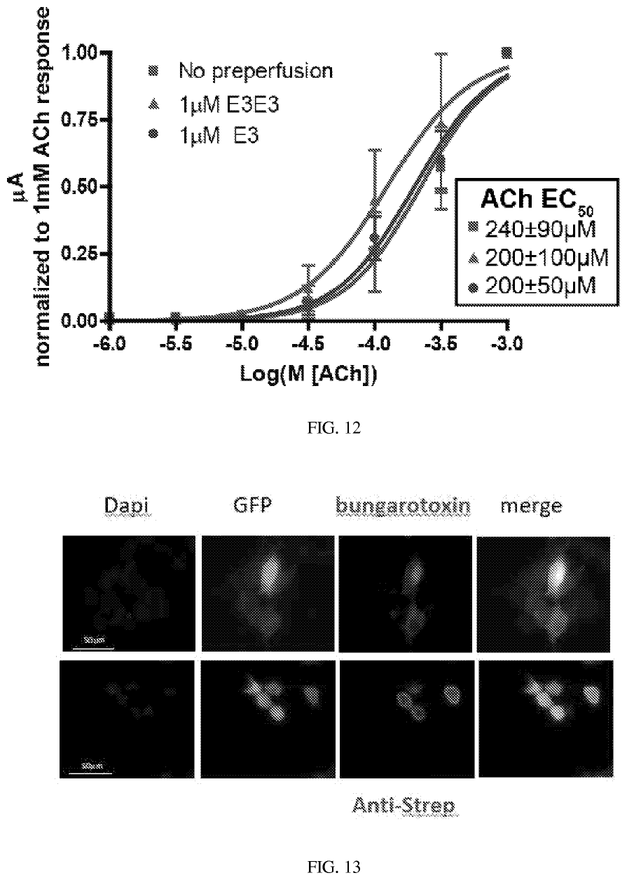

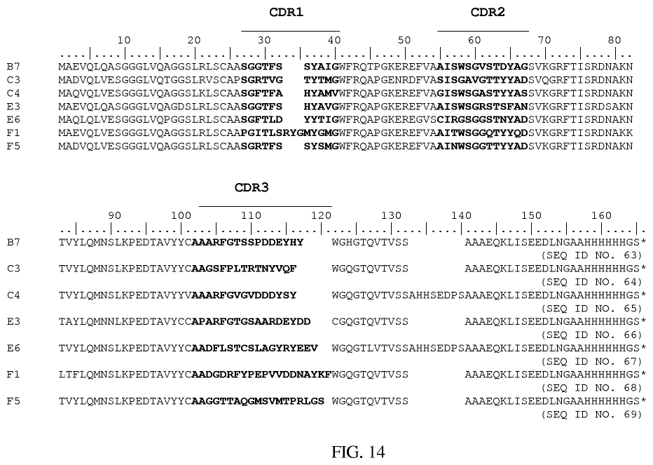

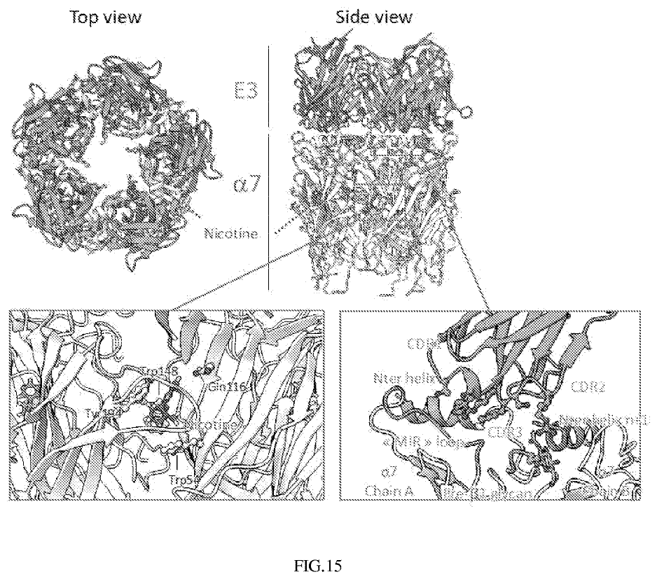

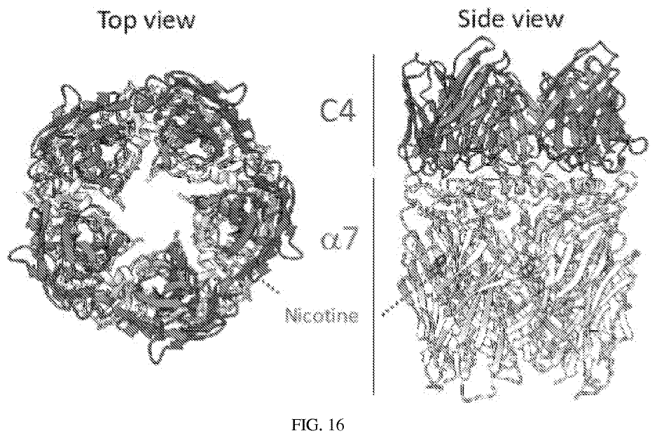

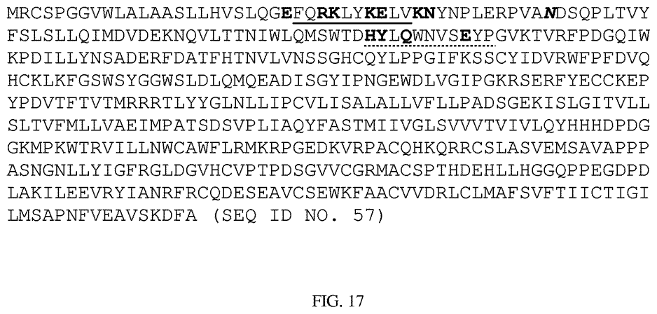

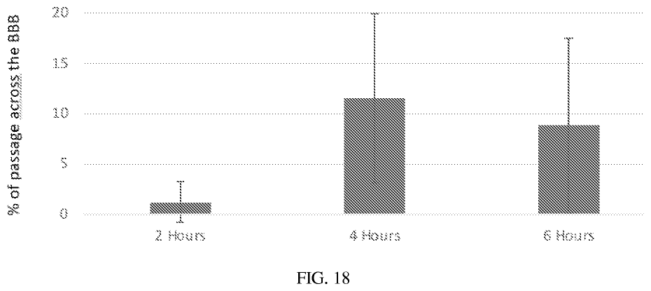

The clones were produced with a C-terminal Myc and His tag (named VHH in FIG. 1) and tested by immunofluorescence (using an anti-Myc secondary antibody) on cells transfected with either α7/5-HT₃A or α7-nAChR. Among the seven VHHs, α7C4 and α7E3 seemed to display specific labeling. Two other variants of α7C4 and α7E3 were generated: 1/ VHH-CSA, carrying a C-terminal cysteine, allowing direct fluorescent labeling to avoid the use of secondary antibodies, and 2/ VHH-Fc, where VHHs are fused at the C-terminus with a human IgG Fc fragment, allowing amplification of the signal using a highly specific secondary anti-human Ig antibody, as well as carrying two VHH domains due to the dimeric nature of the IgG-Fc, putatively increasing binding avidity (FIG. 2). The immunofluorescence of both constructs was carried out against α7-nAChR, the later construct gave robust results (FIG. 3B), and therefore it was used to assess the binding specificity of the constructs.

The VHH-Fc's interaction with α7-, α3β4-, and α4β2-nAChRs was tested using cells transiently transfected with human nAChR subunits either directly fused with or in tandem with an eGFP to visually pinpoint transfected cells: 1/a plasmid coding for the human α7-nAChR followed by an IRES (Internal Ribosome Entry Site) and the eGFP, together with a plasmid coding for the chaperon protein NACHO (Gu et al., 2016), 2/two plasmids coding for the α3-nAChR-IRES-eGFP and β4 (with a C-terminus StrepTagII)-nAChR subunits and 3/a plasmid coding for the β2-nAChR subunit and a plasmid coding for the α4-nAChR subunit directly fused cytoplasmically to an eGFP. Proper expression of the various nAChRs was verified by an anti-StrepTagII antibody for the α3β4-_{StrepTagII}-nAChR, by the GFP fused to α4_{GFP}β2-nAChR, and with an Alexa647 labeled α-bungarotoxin (α-Btx), a competitive and highly specific antagonist, upon expression of α7-nAChR (FIG. 13).

Using the α7C4-Fc and α7E3-Fc and an anti-human IgG to label the transfected and permeabilized cells (FIG. 3C), data show that both α7C4-Fc and α7E3-Fc label cells transfected with α7-nAChR, while no labelling is observed for α3β4- and α4β2-nAChR transfected cells. Similar immunofluorescence experiments on non-permeabilized cells show nice and strong decoration of the plasma membrane by both α7E3-Fc and α7C4-Fc on cells expressing α7-nAChR, compatible with a binding of the VHHs to the ECD (FIG. 3B).

Interestingly, sequence analysis determined that both α7E3 and α7C4 diverge from the classical VHH framework in that 7C4 contains only a single cysteine and thus no disulfide bridge, whereas α7E3 contains four cysteines, forming the canonical bridge between framework region FR1 and FR3 domains, but also an unusual bridge flanking both extremities of the complimentary determining region CDR3 (FIG. 14).

### Example 3. α7E3 is a Potent and Slowly Associating Type I PAM of the α7 nAChR Binding Outside the Orthosteric Site

The functional effect of α7E3 and α7C4 (with a C-terminal Myc and His-tag) were investigated by two-electrode voltage clamp electrophysiology (TEVC) on Xenopus oocytes expressing the hα7-nAChR (FIGS. 5-8). Using 1 mM ACh, in this methodology, evokes robust currents characterized by a fast onset of activation, culmination at a peak, and a slower decay of activation whereupon receptor desensitization through prolonged agonist application overcomes newly activating receptors, until the agonist is rinsed, and the receptor returns to its resting state. An EC₅₀ of 240+/−90 μM was determined, and therefore the effects of the VHHs were investigated at a concentration of 30 μM ACh, corresponding approximately to an EC₁₀ concentration to ensure maximal sensitivity of the system.

α7E3 does not elicit any current when applied alone (FIG. 6). In a first series of experiments, α7E3 was applied during an application of ACh (“post-perfusion condition”, FIG. 4). At 800 nM, α7E3 elicits a clear increase of the current, that appears relatively slowly, followed by a time-dependent decrease, indicating that the VHH favors the activation process but does not alter the downstream process of desensitization, and this potentiating effect is dose-dependent. To further investigate the kinetics by which α7E3 modulates the function, several dose-response recordings were performed where E3 is directly co-applied with ACh (“co-perfusion condition”, FIG. 5), or where it is perfused before an ACh/α7E3 mix for periods of 10, 30, 60 and 120 s (“pre-perfusion conditions”, FIG. 6). Longer pre-perfusion conditions generated significantly larger potentiation, especially at low VHH concentrations, indicating that the VHH associates to the receptor with a slow kinetics in the recording conditions. This slow kinetics is thought to be the main reason for the moderate potentiation observed in post-perfusion conditions. Indeed, in these conditions, α7E3 will slowly bind and potentiate the receptor, a process during which a significant fraction of the activated receptors will desensitize, generating a net reduction of the peak current.

Altogether, α7E3 has the hallmarks of a type I PAM, with low nanomolar apparent affinity, and with a slow binding kinetic. Whereas, α7C4 elicited no response on its own nor showed any discernable modification of α7-nAChR function when co-applied with ACh (FIG. 8).

To further document a PAM binding mode, a competition assay with I¹²⁵ labeled α-Btx orthosteric antagonist was performed. HEK293 cells transfected with the hα7-nAChR/5-HT₃A chimeric construct used in panning experiments were suspended and incubated with 300 nM VHH, a concentration where α7E3 elicits a maximal effect in electrophysiology, and compared to 1.1 mM ACh as a control. Scintillation reading of filtered samples after the addition of a saturating concentration of 25 nM of I¹²⁵-α-Btx was added to this mixture showed that there is no competition between either VHH and α-Btx (FIG. 9). This indicate that neither VHH binds to the orthosteric binding sites, and therefore bind elsewhere within the ECD.

### Example 4. Bivalent α7E3E3 is a Quasi-Irreversible Type I PAM

To increase the affinity and avidity of α7E3 against hα7-nAChR, a bivalent construct was generated, where two α7E3 VHHs are separated by a 16-residue flexible linker (FIG. 1). Strikingly, the potentiating effect of α7E3E3 did not vanish upon prolonged wash out of the oocyte (tested for a period of up to 40 min, FIG. 10), whereas the effect of α7E3 totally vanished upon 5 min of washing (FIG. 6). α7E3E3 is at least as efficient as α7E3 to potentiate the ACh-elicited currents, but evaluation of a concentration dependent curve is rendered moot with the impossibility of washing out the construct. Thus, the avidity conferred by the bifunctional character makes α7E3E3 a quasi-irreversible potentiator in the oocyte recording system.

### Example 5. α7E3 and α7E3E3 Binding Generate a Biphasic ACh Dose-Response Curve with Strong Potentiation at Low ACh Concentrations

The potentiating effect of α7E3 and α7E3E3 were evaluated over a broad range of concentrations of ACh (FIG. 12). The ACh dose-response curve in the absence of VHH is well fitted by a sigmoid. Data show that the VHHs do not significantly change the ACh EC₅₀ of activation.

### Example 6. α7C4 Acts as a SAM that Competes with α7E3 Binding

Current traces, using a 60 s pre-perfusion procedure, with a mixture of α7C4 at 1.5 μM and α7E3 at 150 nM yields an ACh-gated current identical to that in the absence of VHH (FIG. 8). This show that α7C4 does not generate discernable current in the absence of ACh, but that it totally abolishes the potentiation of the currents elicited by co-perfusion of ACh with α7E3. These data show that α7C4 does bind to the receptor in the oocyte system and acts as a silent allosteric modulator (SAM) that binds to a site overlapping, at least partially, with the potentiating site of α7E3. It is noteworthy that comparison of the sequence of α7E3 and α7C4 show significant conservation at CDR1, CDR2 and half of CDR3, further arguing for overlapping binding sites.

### Example 7. Analysis of the E3 and C4 Common Epitope

C4 and E3 bind to α7 in a highly similar manner (FIGS. 15-16), their CDRs contacting the top-ECD platform formed by the principal α7 subunit down below (with a ˜500 Å2 surface of interaction), and the complementary α7 subunit (˜300 Å2). On the principal subunit, it is formed by the C-terminal half of the top a helix that faces the vestibule (N-ter helix), and by the long β2-β3 loop, also called Main Immunogenic Region (MIR) 15, on the outer side. On the complementary subunit, it is formed by the N-terminal half of the top α-helix (N-ter helix).
Both nanobodies display unique disulfide bond patterns. First, E3 harbours the “canonical” disulfide bond that links the strands that just precedes the CDR1 and CDR3, which is missing in C4. Second, the CDR3 of E3 is longer by two residues, and is bent upwards by the presence of an extra disulfide bond between the end of the CDR3 and the second β-strand of the framework region 3 (FR3). In known nanobodies featuring a long CDR3, a supplementary disulfide bond rigidifying the structure is common. For both nanobodies, conserved side chains from the three CDRs (His32_{CDR1}, Trp54_{CDR2}, Arg101_{CDR3} and Phe102_{CDR3}, C4 numbering) are directly plunging into a groove bordered by the helix and MIR elements. Both nanobodies elicit a complex pattern of interactions with α7. Although the sets of interactions are different in E3 and C4, they involve conserved residues from the CDR1, CDR2 and first half of CDR3 (Gly27_{CDR1}, Tyr33_{CDR1}, Trp54_{CDR2}, Arg101_{CDR3}, Phe102_{CDR3}, C4 numbering). They involve also non-conserved residues from the second half of the CDR3, where C4 residues Asp109 and Ser111 are engaged in an extensive set of interactions with the principal N-terminal helix, while much fewer contacts are found in E3. In addition, E3 shows a unique interaction between the Arg56_{CDR2} guanidinium and both the main chain carbonyl and the first glycan grafted on α7Asn23 (pre-β1), an interaction absent in C4 that harbours an alanine in place of the arginine at this position. Overall, the CDR3 is the main interacting loop, contacting the MIR and the N-terminal helices of both subunits.
It was further investigated the contribution of specific α7 motifs to VHH binding by mutagenesis of α7FLcryo. On the one hand, the charged residues of the N-terminal helix of α7 (R4Q K5Q K9Q triple mutant and E9Q K12Q N13A triple mutant) were mutated and on the other hand the MIR residues were replaced to the aligned MIR of the α3-nAChR which does not bind C4 and E3. Finally, N-linked glycosylation was prevented by mutating the serine of the Asn-X-Ser motif (Ser25Ala). It was performed immunofluorescence assays in non-permeabilized HEK293 cells. Immunolabelling of the extracellular C-terminal Rho1D4 tag shows that the MIR and glycosylation mutants, but not the N-terminal helix mutants are expressed at the cell surface. Immunolabelling with E3 and C4, using nanobodies fused to the heavy chain fragment of a human IgG show clear labelling of cells expressing the WT and glycosylation mutant, but no labelling of the MIR mutant. This confirms that the MIR is essential for the VHH binding, while the glycosylation on Asn23 is not mandatory for E3 high-affinity binding.

### Examples 8-15: Material and Methods

### Example 8. Animal Immunization and Library Construction

All immunization processes were executed according to the French legislation and in compliance with the European Communities Council Directives (2010/63/UE, French Law 2013-118, Feb. 6, 2013). The Animal Experimentation Ethics Committee of Pasteur Institute (CETEA 89) approved this study (2020-27412). We subcutaneously injected an adult alpaca at days 0, 21, and 28 with approximately 10⁸ α7/5-HT₃A chimera-transfected HEK293 cells mixed with Freund complete adjuvant for the first immunization and with Freund incomplete adjuvant for the following immunizations. Repetitive immunogen administrations were applied to stimulate a strong immune response. A blood sample of about 250 mL of the immunized animal was collected and a Phage-Display library in a pHEN6 phagemid vector of about 2·10⁸ different clones was prepared as described before (Gransagne et al., 2022).

### Example 9. Molecular Biology

The plasmids for all nAChR constructs were human (h). pcDNA3.1+ vector was used for all IRES containing constructs. The pMT3 vector was used for the eGFP alone and the hα4-nAChR which contained an ICD linked GFP (hα4GFP); where an eGFP sequence was inserted in the cytoplasmic domain between SCK₃₉₅/S₃₉₆PS following the mouse α4-nAChR construct created by Nashmi et al. (J Neurosci, 2003). The pCMV6-XL5 vector was used for the NACHO construct (Gu et al, Neuron 2016). For hα7, hα3, and hβ4_{StrepTagII} (with a C-terminal StrepTagII), the sequence was followed by an internal ribosome entry site (IRES) element and then the coding sequence of eGFP or in the case of hα7 it was also followed by the coding sequence for NACHO used in two-electrode voltage clamp experiments.

VHH-Library preparation. 100 mL bloodletting samples of the immunized alpaca in EDTA-coated tubes were collected and inverted twice to inhibit coagulation. Histopaque-1077 (Roche) was employed to separate B lymphocytes according to the manufacturer's instructions. mRNA was extracted, following the RNeasy minikit (Qiagen) protocol, from isolated lymphocytes and its purity verified using the Agilent RNA 6000 Nano Assay system.

This mRNA was subsequently reverse-transcripted to generate a diverse cDNA library. This cDNA was then amplified by overlap extension PCR which allow for the isolation of the ˜400 bp VHH domain. These PCR fragments are digested using SfiI and NotI and ligated into the pHEN6 phagemid (Conrath et al. Antimicrobial Agents and Chemotherapy, 2001).

Synthetic genes of the anti-α7 VHH derivatives were designed and purchased from EurofinsGenomics (Ebersberg, Germany).

### Example 10. Cell Culture

HEK293 and VERO cells were culture in high glucose Dulbecco's Modified Eagle Medium supplemented with 110 mg/L sodium pyruvate and 862 mg/L of L-alanyl-glutamine as well as 10% fetal bovine serum (DMEM-FBS) with or without the addition of an antibiotic mixture of penicillin/streptomycin (10 U/ml and 10 μg/ml respectively) at 37° C. with 5% CO₂.

### Example 11. Biopanning by Phage-Display on Cells

To produce VHH-phages, 100 μl of library was inoculated into 100 ml of 2×YT medium (Tryptone 16 g/L, Yeast Extract 10 g/L, NaCl 5 g/L, pH 7) supplemented with ampicillin (100 μg/ml) and Glucose (1%), culture was grown at 37° C. under shaking at 200 rpm until OD₆₀₀ (optical density) reached ˜0.5-0.6, recombinant nanobodies-displaying phages were rescued with 2×10¹¹ PFU of KM13 Helper phage (New England Biolabs). Rescued phages were suspended in 1 ml of Phosphate Buffered Saline (PBS, Gibco) supplemented with Bovine Serum Albumin (BSA, Sigma-Aldrich) at a concentration of 1% (PBS-BSA).

To get α7-specific nanobodies, each round of library selection consisted of two depletion steps on non-transfected HEK293 cells, followed by a depletion step on non-transfected Vero cells as negative targets and a positive selection on Vero cells transfected with hα7/m5-HT₃A chimera. Selection steps were carried out at 4° C. for 1 h with gentle shaking in a total volume of 5 ml.

In the first round of panning, 0.5 mL of produced VHH-phages was added to 4.5 mL of PBS-BSA solution as a phage panning medium. 1×T75 confluent HEK cells (˜10⁷) were washed with PBS and resuspended to individual cells, suspension was centrifuged at 2000 rpm at 4° C. for 15 minutes. Cells were resuspended with 5 mL phage panning medium for the first-depletion round, excluding irrelevant binding phages from the library, the suspension was then centrifuged (same conditions as above), and the supernatant phages were recovered. This depletion step was repeated and then further repeated on Vero cells (5 mL, ˜27) to eliminate non-specific nanobodies directed against common membrane proteins. Phages were then finally incubated with hα7/m5-HT₃A chimera-transfected Vero cells to select target-specific VHH-phages. After incubation, suspension was centrifuged (as above) and washed 10 times with PBST (PBS with 0.01% Tween20) (each time 10 minutes) followed by 2 washes with PBS to remove Tween. The cells were then lysed with 1 mL of 100 mM triethylamine TEA (Sigma-Aldrich) for 5 minutes at 4° C. and pH was neutralized with 0.5 mL of 1M Tris (pH 7.4). Eluted phages (in TEA lysate) were amplified by infecting exponentially growing E. coli TG1 at OD₆₀₀ (optical density) of ˜0.5 to propagate phages for the next round of selection.

### Example 12. Cell-ELISA to Detect α7-Specific Nanobodies

96 isolated clones from the second and the third rounds of panning each and were tested for VHH-phage specific binding to α7-nAChR in cell-ELISA.

hα7/m5-HT₃A chimera-transfected Vero cells (10⁵ cells/mL) were coated onto 96-well plates pretreated with poly-D-Lysine (Sigma-Aldrich) according to manufacturer's recommendations, a negative control (non-transfected Vero cells) was prepared under the same conditions. Cells were then fixed by addition of paraformaldehyde (PFA, Sigma-Aldrich) 4% (w/v) in PBS for 20 min, culture were then rinsed three times with PBS and treated with 3% (w/v) hydrogen peroxide (BioRad) in PBS for 5 min to minimize endogenous peroxidase activity, and finally washed twice in PBS. Plates were then blocked with PBS-BSA for 1 h at 37° C.

VHH-phage unique clones were separately grown in 300 μl of 2×YT medium supplemented with ampicillin (100 μg/ml) in a 96-deep wells plate, unique VHH-phage's production was induced by co-infection with the Helper Phage and culture was left overnight at 30° C. under shaking. Supernatant was retrieved by centrifugation (2500 rpm/20 minutes at 4° C.) and produced unique phages were tested in parallel for their specific binding on hα7/5-HT₃A chimera-transfected Vero cells and on non-transfected ones as a control. Supernatant was diluted to 1/5 in PBS-BSA, and 100 μl of the dilution were applied to fixed cells in the corresponding wells, after incubation for 1 h at 37° C., plates were washed 3 times with PBST, and bound VHH-phages were labelled by addition of anti-M13 antibody [HRP] (SinoBiologicals). After 5 washes with PBST, HRP substrate reagent (OrthoPhenyleneDiamine, Sigma), prepared according to manufacturer's recommendation, was added. Reaction was stopped by addition of 50 μl per well of 3M HCl, absorbance of samples was measured at 492 nm in a spectrophotometer microplate reader (Sunrise, Tecan). Samples with absorbance ratio (positive/control) exceeding 3 were considered as α7-positive clones, they were sequenced, and sequences were analyzed.

### Example 13. Anti-α7 VHH Engineering, Expression and Labelling

The pHEN6 vector used for library construction allows direct bacterial periplasmic expression and purification of selected nanobodies with a cMyc tag and a 6×His tag at their C-terminal (FIG. 1 VHH).

The gene coding for a monovalent VHH with a 6×His tag in the N-terminal and an extra C-terminal Cys-Ser-Ala motif (CSA) enables site-specific labelling of the VHH via maleimide (FIG. 1 VHH-CSA) (Vandesquille et al., 2017). The gene for the bivalent derivative is composed of two molecules of the same VHH linked together by a flexible (G4S)₃ linker, a cMyc tag and a 6×His tag at the C-terminal (FIG. 1 VHH-VHH). Genes were cloned into the pHEN6 vector for the periplasmic expression in the E. coli strain BL21 (DE3). Proteins were produced as periplasmic components in 1 L of the NZY Auto-Induction TB medium (NZytech) according to manufacturer's recommendations and were purified by immobilized metal affinity chromatography on a HiTrap TALON® crude 1 mL column (Cytiva). After extensive washings with PBS containing 150 mM NaCl (PBS/NaCl), proteins were eluted in PBS/NaCl buffer supplemented with 500 mM imidazole. Bacterial production yields varied from 1-12 mg/L of culture.

Alternatively, nanobodies' engineered genes were cloned into a pFUSE-derived vector (Invivogen), this vector is harboring a human IgG1 Fc domain, consequently the VHH is expressed as a Fc-fusion bivalent antibody. The vector was used to transform EXPI293F mammalian cells (ThermoFisher), and protein expression was carried out according to manufacturer's recommendations. Protein was then purified from the expression medium by affinity chromatography on a 1 mL protein G column (Cytiva), after sample application column was washed with 20 column volumes of PBS and protein was subsequently eluted with 10 column volumes of PBS supplemented with 0.1 M of Glycine (pH=2.3), production yields were above 25 mg/L of culture.

Site specific labelling of the engineered VHH was done using maleimide ALEXA FLUOR™ 647 C2 or ALEXA FLUOR™ 488 (ThermoFisher). Briefly, the anti-α7 VHH with additional CSA motif was submitted to a mild reduction by adding 10 molar excesses of tris(2-carboxyethyl) phosphine (TCEP) (Sigma-Aldrich) at 25° C. for 30 minutes. Reduced protein was then incubated for 2 hours with 20 molar excess of the Maleimide ALEXA FLUOR dissolved in DMSO. Labelled protein was filter-dialyzed against 10 L of PBS using a 3K-CutOff SLIDE-A-LYZER™ Dialysis Cassette (ThermoFisher), labelling quality was assisted by Mass Spectrometry (Li et al., 2016).

### Example 14. Immunofluorescence

HEK293 cells were cultured on poly-D-lysine coated (Sigma-Aldrich), coated according to manufacturer's recommendations, glass coverslips. These cells were transfected using 10 μg DNA and the JETPRIME transfection reagent (Polyplus) according to manufacturer instructions. 48 h-36 h after transfection, cells were fixed with 4% PFA and permeabilized with ethanol/methanol if necessary. Non-specific binding was blocked with 10% BSA in PBS. The nanobodies were diluted to 5 μg/ml in PBS-BSA and incubated 2 h at room temperature. Anti-StrepTagII antibody and α-bungarotoxin-ALEXA647 (ThermoFisher) were diluted in PBS-BSA. Anti-human IgG and anti-mouse IgG coupled to ALEXA647 (ThermoFisher) were diluted in PBS-BSA. Coverslips were mounted on slides after DAPI staining and visualized using epi-fluorescence at constant exposure times. All experiments were reproduced ≥4 times.

### Example 15. Two-Electrode Voltage-Clamp (TEVC) Electrophysiology

Xenopus laevis oocytes (EcoCyte Bioscience, Germany and Centre de Resources Biologiques—Rennes, France) kept in Barth solution buffer (87.34 mM NaCl, 1 mM KCl, 0.66 mM NaNO3, 0.75 mM CaCl2, 0.82 mM MgSO4, 2.4 mM NaHCO₃, 10 mM HEPES pH 7.6) were nucleus injected with ˜5-8 ng of hα7-IRES-NACHO and ˜1 ng eGFP plasmids and stored at 16° C.-18° C. for 48-96 hours.

Current recordings were performed using: Axon Digidata 1550A digitizer (Molecular Devices), Axon Instruments GeneClamp 500 amplifier (Molecular Devices), a self-built automated voltage control perfusion system which controls an 8-way and 12-way electric rotary valve (Bio-Chem Fluidics) both connected to a 2way 4-port electric rotary valve (Bio-Chem Fluidics) which was flush with the recording chamber allowing for rapid solution application and clean solution exchange, and pClamp 10.6 software (Molecular Devices). Electrode pipettes were pulled using a Narishige PC-10 puller. GFP positive oocytes were voltage-clamped at −60 mV and perfused by Ringer's solution buffer (100 mM NaCl, 2.5 mM KCl, 10 mM HEPES, 2 mM CaCl2, 1 mM MgCl2, pH 7.3), with sampling at 5 kHz. Acetylcholine (ACh) and size-exclusion purified VHH's were diluted in Ringer's buffer as specified. ClampFit 10.6 (Molecular Devices) was used for trace analysis and GraphPad Prism 4 (GraphPad Software) for data plotting and statistical analysis. Data are shown as mean±standard deviation, where each n represents a different oocyte.

The mechanical turning of the valves in the perfusion system produces a noise seen during the recordings, which was not filtered out, allowing for easy recognition of solution exchange times. The main valve allows for one solution to flow to the recording chamber and at the same time the other connected valve, if open, flows to waste. The main 2way/4-port valve exchange time was about 200 ns and there is around a 500 ns delay between the end of the exchange and arrival of the solution to the oocyte. Solution to be perfused to the chamber was started at least 4 s before in the direction of waste to ensure proper rinsing of valve tubing. All recordings contained a five-minute start to start wash time between sweeps.

“Pre-perfusion” protocol: The flow of the denoted concentration of VHH applied 6 s after the start of the sweep to the recording chamber allowing for a proper analysis of the leak/background current. 10-120 s later the main valve was switched to flow a solution that contained the same concentration of VHH with 30 μM ACh (or the denoted concentration in the case of ACh dose-response curve). The valve flowing the VHH alone solution (now to waste) was switched to Ringer's solution shortly after. Five seconds later the main valve was changed back to flow the Ringer's solution to the chamber and the other side valve was closed with the sweep finishing recording 8 s later.

“Purely pre-perfusion” protocol: Once it was determined that the VHH's had a slow on and offset time in order to conserve product and simplify solutions, the pre-perfusion protocol was changed to have only a 30 μM ACh solution applied for the five second activation after pre-perfusion of VHH in Ringer's solution, rather than the mixture of ACh and VHH described above.

“Co-perfusion” protocol: The flow of denoted concentrations of VHH mixed with 30 μM ACh was applied 10 s after the start of the sweep for a duration of 5 s, followed by 15 s of recorded wash.

“Post-perfusion” protocol: In order to properly switch solutions just after the on-set of desensitization there was not enough time to rinse solutions and therefore use the main valve for switching. As a result, the solution was switched between 30 μM ACh to 30 μM ACh with the denoted concentration of VHH in the secondary valve where the delay of solution arrival is around 1.2 s. Therefore, the main valve was switched to flow 30 μM ACh 10 s after the start of the sweep, with the secondary valve switching to a solution of 30 μM ACh with VHH 0.5 s later. This achieved the arrival of the VHH around 2 s (including valve exchange time) after application of ACh alone. The combination was perfused for 6 s before switching back to ACh alone, and the main valve was switched back to Ringer's after 3 s effectively creating a 10 s application of 30 μM ACh which included a combination of ACh with VHH for 6 s directly in the middle.

### Example 16. Structure of Nanobodies in Complex with the Human α7-nAChR

The α7 human nAChR was expressed in HEK 293 cells in suspension. Cells were solubilized in buffer containing 1% dodecylmaltoside and the solubilizate subjected to affinity purification. The purified protein was reconstituted into membrane scaffolding protein nanodiscs in the presence of brain lipid extracts, and detergent was removed using biobeads. For cryo-EM experiments, the purified nAChR was incubated with each VHH in the presence of a saturating concentration of nicotine at 100 μM. The preparation was pluged-freezed on UltrAUFoil grids, and imaged on 200 kV or 300 kV TF cryo-microscopes equipped with direct detectors. Data were processed with Relion and CryoSPARC packages and the structures were build using Phenix and Coot.

### REFERENCES

Buchman A L, Awal M, Jenden D, Roch M, Kang S H. J Am Coll Nutr. 2000 November-December; 19 (6): 768-70. doi: 10.1080/07315724.2000.10718076. PMID: 11194530 Clinical Trial. The effect of lecithin supplementation on plasma choline concentrations during a marathon.
Bouzat, C., Lasala, M., Nielsen, B. E., Corradi, J., Esandi, M. del C., 2018. Molecular function of α7 nicotinic receptors as drug targets: α7 nicotinic receptor. J. Physiol. 596, 1847-1861. https://doi.org/10.1113/JP275101
Brams, M., Govaerts, C., Kambara, K., Price, K. L., Spurny, R., Gharpure, A., Pardon, E., Evans, G. L., Bertrand, D., Lummis, S. C., Hibbs, R. E., Steyaert, J., Ulens, C., 2020. Modulation of the Erwinia ligand-gated ion channel (ELIC) and the 5-HT₃ receptor via a common vestibule site. eLife 9, e51511. https://doi.org/10.7554/eLife.51511
Chandley, M. J., Miller, M. N., Kwasigroch, C. N., Wilson, T. D., Miller, B. E., 2009. Increased antibodies for the α7 subunit of the nicotinic receptor in schizophrenia. Schizophr. Res. 109, 98-101. https://doi.org/10.1016/j.schres.2009.01.023
Corringer, P.-J., Galzi, J.-L., Eisele, J.-L., Bertrand, S., Changeux, J.-P., Bertrand, D., 1995. Identification of a New Component of the Agonist Binding Site of the Nicotinic α7 Homooligomeric Receptor. J. Biol. Chem. 270, 11749-11752. https://doi.org/10.1074/jbc.270.20.11749
Craig, P. J., Bose, S., Zwart, R., Beattie, R. E., Folly, E. A., Johnson, L. R., Bell, E., Evans, N. M., Benedetti, G., Pearson, K. H., McPhie, G. I., Volsen, S. G., Millar, N. S., Sher, E., Broad, L. M., 2004. Stable expression and characterisation of a human α7 nicotinic subunit chimera: a tool for functional high-throughput screening. J. Pharmacol. 502, 31-40. Eur. https://doi.org/10.1016/j.ejphar.2004.08.042
Delbart, F., Brams, M., Gruss, F., Noppen, S., Peigneur, S., Boland, S., Chaltin, P., Brandao-Neto, J., von Delft, F., Touw, W. G., Joosten, R. P., Liekens, S., Tytgat, J., Ulens, C., 2018. An allosteric binding site of the α7 nicotinic acetylcholine receptor revealed in a humanized acetylcholine-binding protein. J. Biol. Chem. 293, 2534-2545. https://doi.org/10.1074/jbc.M117.815316
Dong, J., Huang, B., Wang, B., Titong, A., Gallolu Kankanamalage, S., Jia, Z., Wright, M., Parthasarathy, P., Liu, Y., 2020. Development of humanized tri-specific nanobodies with potent neutralization for SARS-COV-2. Sci. Rep. 10, 17806. https://doi.org/10.1038/s41598-020-74761-y
Galzi, J. L., Bertrand, S., Corringer, P. J., Changeux, J. P., Bertrand, D., 1996. Identification of calcium binding sites that regulate potentiation of a neuronal nicotinic acetylcholine receptor. EMBO J. 15, 5824-5832. https://doi.org/10.1002/j.1460-2075.1996.tb00969.x
Gill, J. K., Savolainen, M., Young, G. T., Zwart, R., Sher, E., Millar, N. S., 2011. Agonist activation of α7 nicotinic acetylcholine receptors via an allosteric transmembrane site. Proc. Natl. Acad. Sci. 108, 5867-5872. https://doi.org/10.1073/pnas. 1017975108 Gransagne, M., Aymé, G., Brier, S., Chauveau-Le Friec, G., Meriaux, V., Nowakowski, M., Dejardin, F., Levallois, S., Dias de Melo, G., Donati, F., Prot, M., Brûlé, S., Raynal, B., Bellalou, J., Goncalves, P., Montagutelli, X., Di Santo, J. P., Lazarini, F., England, P., Petres, S., Escriou, N., Lafaye, P., 2022. Development of a highly specific and sensitive VHH-based sandwich immunoassay for the detection of the SARS-COV-2 nucleoprotein. J. Biol. Chem. 298, 101290. https://doi.org/10.1016/j.jbc.2021.101290
Gu, S., Matta, J. A., Lord, B., Harrington, A. W., Sutton, S. W., Davini, W. B., Bredt, D. S., 2016. Brain α7 Nicotinic Acetylcholine Receptor Assembly Requires NACHO. Neuron 89, 948-955. https://doi.org/10.1016/j.neuron.2016.01.018
Hassaine, G., Deluz, C., Grasso, L., Wyss, R., Tol, M. B., Hovius, R., Graff, A., Stahlberg, H., Tomizaki, T., Desmyter, A., Moreau, C., Li, X.-D., Poitevin, F., Vogel, H., Nury, H., 2014. X-ray structure of the mouse serotonin 5-HT₃ receptor. Nature 512, 276-281. https://doi.org/10.1038/nature13552
Hultberg, A., Temperton, N. J., Rosseels, V., Koenders, M., Gonzalez-Pajuelo, M., Schepens, B., Ibañez, L. I., Vanlandschoot, P., Schillemans, J., Saunders, M., Weiss, R. A., Saelens, X., Melero, J. A., Verrips, C. T., Van Gucht, S., de Haard, H. J., 2011. Llama-Derived Single Domain Antibodies to Build Multivalent, Superpotent and Broadened Neutralizing Anti-Viral Molecules. PLOS ONE 6, e17665. https://doi.org/10.1371/journal.pone.0017665
Hurst, R. S., 2005. A Novel Positive Allosteric Modulator of the 7 Neuronal Nicotinic Acetylcholine Receptor: In Vitro and In Vivo Characterization. J. Neurosci. 25, 4396-4405. https://doi.org/10.1523/JNEUROSCI.5269-04.2005
Jovčevska, I., Muyldermans, S., 2020. The Therapeutic Potential of Nanobodies. BioDrugs 34, 11-26. https://doi.org/10.1007/s40259-019-00392-z
Krause, R. M., Buisson, B., Bertrand, S., Corringer, P.-J., Galzi, J.-L., Changeux, J.-P., Bertrand, D., n.d. Ivermectin: A Positive Allosteric Effector of the _7 Neuronal Nicotinic Acetylcholine Receptor 12.
Lasala, M., Corradi, J., Bruzzone, A., Esandi, M. del C., Bouzat, C., 2018. A human-specific, truncated α7 nicotinic receptor subunit assembles with full-length α7 and forms functional receptors with different stoichiometries. J. Biol. Chem. 293, 10707-10717. https://doi.org/10.1074/jbc.RA117.001698
Le Novère, N., Grutter, T., Changeux, J.-P., 2002. Models of the extracellular domain of the nicotinic receptors and of agonist- and Ca²⁺-binding sites. Proc. Natl. Acad. Sci. 99, 3210-3215. https://doi.org/10.1073/pnas.042699699
Li, T., Vandesquille, M., Koukouli, F., Dudeffant, C., Youssef, I., Lenormand, P., Ganneau, C., Maskos, U., Czech, C., Grueninger, F., Duyckaerts, C., Dhenain, M., Bay, S., Delatour, B., Lafaye, P., 2016. Camelid single-domain antibodies: A versatile tool for in vivo imaging of extracellular and intracellular brain targets. J. Controlled Release 243, 1-10. https://doi.org/10.1016/j.jconrel.2016.09.019
Masiulis, S., Desai, R., Uchański, T., Serna Martin, I., Laverty, D., Karia, D., Malinauskas, T., Zivanov, J., Pardon, E., Kotecha, A., Steyaert, J., Miller, K. W., Aricescu, A. R., 2019. GABAA receptor signalling mechanisms revealed by structural pharmacology. Nature 565, 454-459. https://doi.org/10.1038/s41586-018-0832-5
Natarajan, K., Mukhtasimova, N., Corradi, J., Lasala, M., Bouzat, C., Sine, S. M., 2020. Mechanism of calcium potentiation of the α7 nicotinic acetylcholine receptor. J. Gen. Physiol. 152, e202012606. https://doi.org/10.1085/jgp.202012606
Nemecz, Á., Prevost, M. S., Menny, A., Corringer, P.-J., 2016. Emerging Molecular Mechanisms of Signal Transduction in Pentameric Ligand-Gated Ion Channels. Neuron 90, 452-470. https://doi.org/10.1016/j.neuron.2016.03.032
Noviello, C. M., Gharpure, A., Mukhtasimova, N., Cabuco, R., Baxter, L., Borek, D., Sine, S. M., Hibbs, R. E., 2021. Structure and gating mechanism of the α7 nicotinic acetylcholine receptor. Cell 184, 2121-2134.e13. https://doi.org/10.1016/j.cell.2021.02.049
Papke, R. L., Horenstein, N. A., 2021. Therapeutic Targeting of a 7 Nicotinic Acetylcholine Receptors. Pharmacol. Rev. 73, 1118-1149. https://doi.org/10.1124/pharmrev. 120.000097
Papke, R. L., Porter Papke, J. K., 2002. Comparative pharmacology of rat and human α7 nAChR conducted with net charge analysis: Pharmacology of rat and human α7AChR. Br. J. Pharmacol. 137, 49-61. https://doi.org/10.1038/sj.bjp.0704833
Scholler, P., Nevoltris, D., de Bundel, D., Bossi, S., Moreno-Delgado, D., Rovira, X., Møller, T. C., El Moustaine, D., Mathieu, M., Blanc, E., McLean, H., Dupuis, E., Mathis, G., Trinquet, E., Daniel, H., Valjent, E., Baty, D., Chames, P., Rondard, P., Pin, J.-P., 2017. Allosteric nanobodies uncover a role of hippocampal mGlu2 receptor homodimers in contextual fear consolidation. Nat. Commun. 8, 1967. https://doi.org/10.1038/s41467-017-01489-1
Schoof, M., Faust, B., Saunders, R. A., Sangwan, S., Rezelj, V., Hoppe, N., Boone, M., Billesbølle, Christian B., Puchades, C., Azumaya, C. M., Kratochvil, H. T., Zimanyi, M., Deshpande, I., Liang, J., Dickinson, S., Nguyen, H. C., Chio, C. M., Merz, G. E., Thompson, M. C., Diwanji, D., Schaefer, K., Anand, A. A., Dobzinski, N., Zha, B. S., Simoneau, C. R., Leon, K., White, K. M., Chio, U. S., Gupta, M., Jin, M., Li, F., Liu, Yanxin, Zhang, K., Bulkley, D., Sun, M., Smith, A. M., Rizo, A. N., Moss, F., Brilot, A. F., Pourmal, S., Trenker, R., Pospiech, T., Gupta, S., Barsi-Rhyne, B., Belyy, V., Barile-Hill, A. W., Nock, S., Liu, Yuwei, Krogan, N. J., Ralston, C. Y., Swaney, D. L., García-Sastre, A., Ott, M., Vignuzzi, M., QCRG Structural Biology Consortium, Walter, P., Manglik, A., Azumaya, C. M., Puchades, C., Sun, M., Braxton, J. R., Brilot, A. F., Gupta, M., Li, F., Lopez, K. E., Melo, A., Merz, G. E., Moss, F., Paulino, J., Pospiech, T. H., Pourmal, S., Rizo, A. N., Smith, A. M., Thomas, P. V., Wang, F., Yu, Z., Dickinson, M. S., Nguyen, H. C., Asarnow, D., Campbell, M. G., Chio, C. M., Chio, U. S., Diwanji, D., Faust, B., Gupta, M., Hoppe, N., Jin, M., Li, J., Liu, Yanxin, Merz, G. E., Sangwan, S., Tsui, T. K. M., Trenker, R., Trinidad, D., Tse, E., Zhang, K., Zhou, F., Herrera, N., Kratochvil, H. T., Schulze-Gahmen, U., Thompson, M. C., Young, I. D., Biel, J., Deshpande, I., Liu, X., Billesbølle, Christian Bache, Nowotny, C., Smith, A. M., Zhao, J., Bowen, A., Hoppe, N., Li, Y.-L., Nguyen, P., Safari, M., Schaefer, K., Whitis, N., Moritz, M., Owens, T. W., Diallo, A., Kim, K., Peters, J. K., Titus, E. W., Chen, J., Doan, L., Flores, S., Lam, V. L., Li, Y., Lo, M., Thwin, A. C., Wankowicz, S., Zhang, Y., Bulkley, D., Joves, Arceli, Joves, Almarie, Mckay, L., Tabios, M., Rosenberg, O. S., Verba, K. A., Agard, D. A., Cheng, Y., Fraser, J. S., Frost, A., Jura, N., Kortemme, T., Krogan, N. J., Manglik, A., Southworth, D. R., Stroud, R. M., 2020. An ultrapotent synthetic nanobody neutralizes SARS-COV-2 by stabilizing inactive Spike. Science 370, 1473-1479. https://doi.org/10.1126/science.abe3255
Spurny, R., Debaveye, S., Farinha, A., Veys, K., Vos, A. M., Gossas, T., Atack, J., Bertrand, S., Bertrand, D., Danielson, U. H., Tresadern, G., Ulens, C., 2015. Molecular blueprint of allosteric binding sites in a homologue of the agonist-binding domain of the α7 nicotinic acetylcholine receptor. Proc. Natl. Acad. Sci. 112. https://doi.org/10.1073/pnas. 1418289112
Terry, A. V., Callahan, P. M., 2020. α7 nicotinic acetylcholine receptors as therapeutic targets in schizophrenia: Update on animal and clinical studies and strategies for the future. Neuropharmacology 170, 108053. https://doi.org/10.1016/j.neuropharm.2020.108053
Terryn, S., Francart, A., Lamoral, S., Hultberg, A., Rommelaere, H., Wittelsberger, A., Callewaert, F., Stohr, T., Meerschaert, K., Ottevaere, I., Stortelers, C., Vanlandschoot, P., Kalai, M., Van Gucht, S., 2014. Protective Effect of Different Anti-Rabies Virus VHH Constructs against Rabies Disease in Mice. PLOS ONE 9, e109367. https://doi.org/10.1371/journal.pone.0109367
Uchański, T., Pardon, E., Steyaert, J., 2020. Nanobodies to study protein conformational states. Curr. Opin. Struct. Biol. 60, 117-123. https://doi.org/10.1016/j.sbi.2020.01.003
Vandesquille, M., Li, T., Po, C., Ganneau, C., Lenormand, P., Dudeffant, C., Czech, C., Grueninger, F., Duyckaerts, C., Delatour, B., Dhenain, M., Lafaye, P., Bay, S., 2017. Chemically-defined camelid antibody bioconjugate for the magnetic resonance imaging of Alzheimer's disease. mAbs 9, 1016-1027. https://doi.org/10.1080/19420862.2017.1342914
Wang, Hong, Yu, M., Ochani, M., Amella, C. A., Tanovic, M., Susarla, S., Li, J. H., Wang, Haichao, Yang, H., Ulloa, L., Al-Abed, Y., Czura, C. J., Tracey, K. J., 2003. Nicotinic acetylcholine receptor α7 subunit is an essential regulator of inflammation 421, 5.
Wu, J., Liu, Q., Tang, P., Mikkelsen, J. D., Shen, J., Whiteaker, P., Yakel, J. L., 2016. Heteromeric α7β2 Nicotinic Acetylcholine Receptors in the Brain. Trends Pharmacol. Sci. 37, 562-574. https://doi.org/10.1016/j.tips.2016.03.005
Zhao, Y., Liu, S., Zhou, Y., Zhang, M., Chen, H., Eric Xu, H., Sun, D., Liu, L., Tian, C., 2021. Structural basis of human α7 nicotinic acetylcholine receptor activation. Cell Res. 31, 713-716. https://doi.org/10.1038/s41422-021-00509-6

1. An isolated antibody that is an allosteric modulator of alpha7 nAChR.

2. The antibody of claim 1, wherein the antibody is a single domain antibody, preferably a VHH.

3. The antibody of claim 2, wherein the single domain antibody, preferably the VHH, comprises a CDR1 having the amino acid sequence SGFTFAHYAMV (SEQ ID NO: 18) or SGGTFSHYAVG (SEQ ID NO: 19) or XGXTFXHYAXX (SEQ ID NO: 14).

4. The antibody of claim 2 or 3, wherein the single domain antibody, preferably the VHH comprises a CDR2 having the amino acid sequence GISWSGASTYYAS (SEQ ID NO: 28) or AISWSGRSTSFAN (SEQ ID NO: 29) or XISWSGXSTXXAX (SEQ ID NO: 24).

5. The antibody of any of claims 2-4, wherein the single domain antibody, preferably the VHH comprises a CDR3 having the amino acid sequence ARFGVGVDDDYSY (SEQ ID NO: 35) or ARFGTGSAARDEYDD (SEQ ID NO: 36).

6. The antibody of claim 2, wherein the single domain antibody, preferably the VHH, comprises the following complementary determining regions (CDR):
CDR1 having the amino acid sequence selected from SEQ ID NO: 13-22 and variants thereof having no more than 2 mismatches compared to SEQ ID NO: 13-22;
CDR2 having the amino acid sequence selected from SEQ ID NO: 23-32 and variants thereof having no more than 2 mismatches compared to SEQ ID NO: 23-32; and
CDR3 having the amino acid sequence selected from SEQ ID NO: 33-39 and variants thereof having no more than 2 mismatches compared to SEQ ID NO: 33-39.

7. The antibody of any of claims 2 to 6, wherein the VHH comprises the amino acid sequence selected from the group consisting of SEQ ID NO: 1-12.

8. The antibody of any of claims 2 to 7, wherein the VHH comprises the amino acid sequence of VHH α7E3 (SEQ ID NO: 4) or VHH α7C4 (SEQ ID NO: 3).

9. The antibody of any of claims 2 to 8, wherein the antibody is multivalent.

10. The antibody of any one of claims 2 to 9, wherein the antibody is bivalent.

11. A multimeric construct comprising the antibody of any one of claims 1 to 8 covalently linked to at least one second polypeptide.

12. The antibody of any one of claims 2 to 10 or the multimeric construct of claim 11, wherein the antibody or the multimeric construct is engineered to cross the blood-brain barrier.

13. The antibody of any one of claims 2 to 10 and 12 or the multimeric construct of claim 11 or 12, wherein the antibody or the multimeric construct is a fusion between the VHH as defined in any one of claims 2-8 and a second VHH targeting the transferrin receptor.

14. A nucleic acid encoding the antibody of any of claims 1-10 and 12-13 or the multimeric construct of claim 11 or 12-13.

15. A vector comprising the nucleic acid of claim 14.

16. A composition comprising the antibody of any one of claims 1 to 10 and 12-13 or the multimeric construct of claim 11 or 12-13 and a pharmaceutically acceptable vehicle.

17. The antibody of any of claims 1-10 and 12-13 or the multimeric construct of claim 11 or 12-13 or the composition of claim 16 for use in a method of treatment.

18. The antibody of any one of claims 1 to 10 and 12-13 or the multimeric construct of claim 11 or 12-13 or the composition of claim 16 for use in a method of treatment of cognitive disorders.

19. The antibody of any one of claims 1 to 10 and 12-13 or the multimeric construct of claim 11 or 12-13 or the composition of claim 16 for use in a method of treatment of a disease selected from the group consisting of Alzheimer's disease, Parkinson's disease and schizophrenia.

20. A detection agent comprising the antibody of any one of claims 1 to 10 and 12-13 or the multimeric construct of claim 11 or 12-13 and a label.

21. An in vitro method for the detection of alpha7 nAChR comprising the step of:
providing a detection agent comprising the antibody of any one of claims 1 to 10 and 12-13 or the multimeric construct of claim 11 or 12-13;
providing a biological sample;
contacting the detection agent with the biological sample; and
visualizing the antigen-detection agent complexes formed.


SEQUENCE LISTING (99 sequences)

SEQ ID NO: 1 - protein, 125 aa, synthetic construct
MAEVQLQASGGGLVQAGGSLRLSCAASGGTFSSYAIGWFRQTPGKEREFVAAISWSGVSTDYAGSVKGRFTISRDNAKNTVYLQMNSLKPEDTAVYYCAAARFGTSSPDDEYHYWGHGTQVTVSS

SEQ ID NO: 2 - protein, 124 aa, synthetic construct
MADVQLVESGGGLVQTGGSLRVSCAPSGRTVGTYTMGWFRQAPGENRDFVASISGAVGTTYYADSVQGRFTISRDNAKNTVYLQMNSLKPEDTAVYYCAAGSFPLTRTNYVQFWGQGTQVTVSS

SEQ ID NO: 3 - protein, 124 aa, synthetic construct
MAQVQLVESGGGLVQAGGSLKLSCAASGFTFAHYAMVWFRQAPGKEREFVAGISWSGASTYYASSVKGRFTISRDNAKNTVYLQMNSLKPEDTAVYYVAAARFGVGVDDDYSYWGQGTQVTVSS

SEQ ID NO: 4 - protein, 126 aa, synthetic construct
MAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSS

SEQ ID NO: 5 - protein, 126 aa, synthetic construct
MAEVQLQRSGGGRVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSS

SEQ ID NO: 6 - protein, 126 aa, synthetic construct
MAEVQLQASGGGLVQAGDSLRLSCAASGGTFSHYAVGWFRQAPGKEREFVAAISWSGRSTSFANSVKGRFTISRDSAKNTAYLQMRRLKPEDTAVYCCAPARFGTGSAARDEYDDCGQGTQVTVSS

SEQ ID NO: 7 - protein, 126 aa, synthetic construct
MAEVQLQRSGGGLVQAGDSLRLSCAASGGTFSHYAVGWFRQAPGKEREFVAAISWSGRSTSFANSVKGRFTISRDSAKNTAYLQMRNLKPEDTAVYCCAPARFGTGSAARDEYDDCGQGTQVTVSS

SEQ ID NO: 8 - protein, 126 aa, synthetic construct
MAEVQLQRSGGGLVQAGDSLRLSCAASGGTFSHYAVGWFRQAPGKEREFVAAISWSGRSTSFANSVKGRFTISRDSAKNTAYLQMRNLKPEDTAVYCCAPARFGTGSAARDEYDDCGQGTRVTVSS

SEQ ID NO: 9 - protein, 126 aa, synthetic construct
  VARIANT 8: note = X is A or R
  VARIANT 13: note = X is L or R
  VARIANT 86: note = X is N or R
  VARIANT 87: note = X is N or R
  VARIANT 121: note = X is Q or R
MAEVQLQXSGGGXVQAGDSLRLSCAASGGTFSHYAVGWFRQAPGKEREFVAAISWSGRSTSFANSVKGRFTISRDSAKNTAYLQMXXLKPEDTAVYCCAPARFGTGSAARDEYDDCGQGTXVTVSS

SEQ ID NO: 10 - protein, 127 aa, synthetic construct
MAQLQLVESGGGLVQPGGSLRLSCAASGFTLDYYTIGWFRQAPGKEREGVSCIRGSGGSTNYADSVKGRFTISRDNAKNTVYLQMNSLKPEDTAVYYCAADFLSTCSLAGYRYEEVWGQGTLVTVSS

SEQ ID NO: 11 - protein, 132 aa, synthetic construct
MAEVQLVESGGGLVQAGGSLRLSCAAPGITLSRYGMYGMGWFRQAPGKEREFVAAITWSGGQTYYQDSVKGRFTISRDNAKKLTFLQMNSLKPEDTAVYYCAADGDRFYPEPVVDDNAYKFWGQGTQVTVSS

SEQ ID NO: 12 - protein, 128 aa, synthetic construct
MADVQLVESGGGLVQAGGSLRLSCAASGRTFSSYSMGWFRQAPGKEREFVAAINWSGGTTYYADSVKGRFTISRDNAKNTVYLQMNSLKPEDTAVYYCAAGGTTAQGMSVMTPRLGSWGQGTQVTVSS

SEQ ID NO: 13 - protein, 11 aa, synthetic construct
  VARIANT 3: note = X is F, G or R
  VARIANT 5: note = X is L, V or F
  VARIANT 6: note = X is S, G,A or D
  VARIANT 7: note = X is S, T , H or Y
  VARIANT 9: note = X is A, T or S
  VARIANT 10: note = X is I, M or V
  VARIANT 11: note = X is M or G
SGXTXXXYXXX

SEQ ID NO: 14 - protein, 11 aa, synthetic construct
XGXTFXHYAXX

SEQ ID NO: 15 - protein, 11 aa, synthetic construct
  VARIANT 3: note = X is F or G
  VARIANT 6: note = X is A or S
  VARIANT 10: note = X is M or V
  VARIANT 11: note = X is M or G
SGXTFXHYAXX

SEQ ID NO: 16 - protein, 11 aa, synthetic construct
SGGTFSSYAIG

SEQ ID NO: 17 - protein, 11 aa, synthetic construct
SGRTVGTYTMG

SEQ ID NO: 18 - protein, 11 aa, synthetic construct
SGFTFAHYAMV

SEQ ID NO: 19 - protein, 11 aa, synthetic construct
SGGTFSHYAVG

SEQ ID NO: 20 - protein, 11 aa, synthetic construct
SGFTLDYYTIG

SEQ ID NO: 21 - protein, 14 aa, synthetic construct
PGITLSRYGMYGMG

SEQ ID NO: 22 - protein, 11 aa, synthetic construct
SGRTFSSYSMG

SEQ ID NO: 23 - protein, 13 aa, synthetic construct
  VARIANT 1: note = X is G or A
  VARIANT 3: note = X is T, S or N
  VARIANT 7: note = X is A, V, G or R
  VARIANT 8: note = X is S, Q or T
  VARIANT 10: note = X is D, Y or S
  VARIANT 11: note = X is Y or F
  VARIANT 12: note = X is Q or A
  VARIANT 13: note = X is G, D S or N
XIXWSGXXTXXXX

SEQ ID NO: 24 - protein, 13 aa, synthetic construct
XISWSGXSTXXAX

SEQ ID NO: 25 - protein, 13 aa, synthetic construct
  VARIANT 1: note = X is G or A
  VARIANT 7: note = X is A or R
  VARIANT 10: note = X is Y or S
  VARIANT 11: note = X is Y or F
  VARIANT 13: note = X is S or N
XISWSGXSTXXAX

SEQ ID NO: 26 - protein, 13 aa, synthetic construct
AISWSGVSTDYAG

SEQ ID NO: 27 - protein, 13 aa, synthetic construct
SISGAVGTTYYAD

SEQ ID NO: 28 - protein, 13 aa, synthetic construct
GISWSGASTYYAS

SEQ ID NO: 29 - protein, 13 aa, synthetic construct
AISWSGRSTSFAN

SEQ ID NO: 30 - protein, 13 aa, synthetic construct
CIRGSGGSTNYAD

SEQ ID NO: 31 - protein, 13 aa, synthetic construct
AITWSGGQTYYQD

SEQ ID NO: 32 - protein, 13 aa, synthetic construct
AINWSGGTTYYAD

SEQ ID NO: 33 - protein, 16 aa, synthetic construct
AAARFGTSSPDDEYHY

SEQ ID NO: 34 - protein, 15 aa, synthetic construct
AAGSFPLTRTNYVQF

SEQ ID NO: 35 - protein, 15 aa, synthetic construct
AAARFGVGVDDDYSY

SEQ ID NO: 36 - protein, 17 aa, synthetic construct
APARFGTGSAARDEYDD

SEQ ID NO: 37 - protein, 18 aa, synthetic construct
AADFLSTCSLAGYRYEEV

SEQ ID NO: 38 - protein, 20 aa, synthetic construct
AADGDRFYPEPVVDDNAYKF

SEQ ID NO: 39 - protein, 19 aa, synthetic construct
AAGGTTAQGMSVMTPRLGS

SEQ ID NO: 40 - protein, 156 aa, synthetic construct
MGSSHHHHHHAAAAEVQLQRSGGGRVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSCSA

SEQ ID NO: 41 - protein, 156 aa, synthetic construct
MGSSHHHHHHAAAAEVQLQASGGGLVQAGDSLRLSCAASGGTFSHYAVGWFRQAPGKEREFVAAISWSGRSTSFANSVKGRFTISRDSAKNTAYLQMRRLKPEDTAVYCCAPARFGTGSAARDEYDDCGQGTQVTVSSGGGGSGGGGSGGGGSCSA

SEQ ID NO: 42 - protein, 156 aa, synthetic construct
MGSSHHHHHHAAAAEVQLQRSGGGLVQAGDSLRLSCAASGGTFSHYAVGWFRQAPGKEREFVAAISWSGRSTSFANSVKGRFTISRDSAKNTAYLQMRNLKPEDTAVYCCAPARFGTGSAARDEYDDCGQGTQVTVSSGGGGSGGGGSGGGGSCSA

SEQ ID NO: 43 - protein, 156 aa, synthetic construct
MGSSHHHHHHAAAAEVQLQRSGGGLVQAGDSLRLSCAASGGTFSHYAVGWFRQAPGKEREFVAAISWSGRSTSFANSVKGRFTISRDSAKNTAYLQMRNLKPEDTAVYCCAPARFGTGSAARDEYDDCGQGTRVTVSSGGGGSGGGGSGGGGSCSA

SEQ ID NO: 44 - protein, 449 aa, synthetic construct
MGSSHHHHHHSSGLVPRGSAAAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSQVQLQESGPGLVKPSETLSLTCTVSAGSISSTSTSYYWGWIRQSPGKGLEWIGSIYYSGRTYYNPSLKSRVTISVDRPNNQFSLKVSSVTAADSAVYYCARHRRVLLWIGELLDDYDRDVWGQGTMVTVSSGGGGSGGGGSGGGGSCSA

SEQ ID NO: 45 - protein, 370 aa, synthetic construct
MGSSHHHHHHAAAAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSAAARSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 368 aa, synthetic construct
MGSSHHHHHHAAAAQVQLVESGGGLVQAGGSLKLSCAASGFTFAHYAMVWFRQAPGKEREFVAGISWSGASTYYASSVKGRFTISRDNAKNTVYLQMNSLKPEDTAVYYVAAARFGVGVDDDYSYWGQGTQVTVSSAAARSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 129 aa, synthetic construct
QVQLQESGPGLVKPSETLSLTCTVSAGSISSTSTSYYWGWIRQSPGKGLEWIGSIYYSGRTYYNPSLKSRVTISVDRPNNQFSLKVSSVTAADSAVYYCARHRRVLLWIGELLDDYDRDVWGQGTMVTV

SEQ ID NO: 48 - protein, 211 aa, synthetic construct
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 453 bp, synthetic construct
  source 1..453: mol_type = other DNA
atggccgaggtccagctgcaggcgtctgggggaggattggtgcaggctggggactctctgagactctcttgtgcagcctctggaggcaccttcagtcactatgccgtgggctggttccgccaggctccagggaaggagcgtgagttcgtagcagctattagctggagtggtcgtagcacaagctttgcaaactccgtgaagggccgattcaccatctccagagacagcgccaagaatacggcctatctacaaatgaacaacctgaaacctgaggacacggccgtttattgttgtgcaccagcccgatttggtactggatcagcggcgcgggatgagtatgacgactgcggccaggggacccaggtcaccgtctcctcagcggccgcagaacaaaaactcatctcagaagaggatctgaatggggccgcacatcaccaccatcaccatgggagc

SEQ ID NO: 50 - DNA, 471 bp, synthetic construct
  source 1..471: mol_type = other DNA
atggctcaggtgcagctcgtggagtccgggggaggattggtgcaggctgggggctctctgaaactctcctgtgcagcctctggattcaccttcgctcactatgccatggtctggttccgccaggctccagggaaggagcgtgagtttgtagcaggcattagctggagtggtgctagcacatactatgcaagctccgtgaagggccgattcaccatctccagagacaacgccaagaacacggtgtatctgcaaatgaacagcctgaaacctgaggacacggccgtttattacgttgcagcggctaggtttggggttggggtcgacgatgactattcctactggggccaggggacccaggtcaccgtctcctcagcgcaccacagcgaagaccccagcgcggccgcagaacaaaaactcatctcagaagaggatctgaatggggccgcacatcaccaccatcaccatgggagc

SEQ ID NO: 51 - DNA, 1386 bp, synthetic construct
  source 1..1386: mol_type = other DNA
ccatgggcagcagccatcatcatcatcatcacagcagcggcctggtgccgcgcggcagcgctgctgccgaggtccagctgcaggcgtctgggggaggattggtgcaggctggggactctctgagactctcttgtgcagcctctggaggcaccttcagtcactatgccgtgggctggttccgccaggctccagggaaggagcgtgagttcgtagcagctattagctggagtggtcgtagcacaagctttgcaaactccgtgaagggccgattcaccatctccagagacagcgccaagaatacggcctatctacaaatgaacaacctgaaacctgaggacacggccgtttattgttgtgcaccagcccgatttggtactggatcagcggcgcgggatgagtatgacgactgcggccaggggacccaagtcaccgtctcctcagggggcggaggtagtggtggaggtggaagtggaggtggggggtctggtgggggcgggtccgaagttcaattgcaggctagtggcggcggtttagtgcaggccggtgactctctccgcctgtcctgcgcggcatcgggcggcacattcagccattatgccgtgggctggtttcgccaggcaccgggcaaggagcgtgaattcgtcgctgccatttcgtggagtggccggtcgactagctttgcaaacagcgttaaaggccgatttaccatctcacgcgactccgccaaaaacacggcgtacctgcagatgaataatctgaaaccagaggatacggcggtatattgctgtgctccggcgcgtttcggtaccggtagcgcggcgcgtgatgaatacgatgattgtggccagggtacccaggtcacagtgtcaagcggcggcggtggaagtggcgggggaggcagcggtggaggtggatcccaggtgcagctgcaggagagcggccccggcctggtgaagcccagcgagaccctgagcctgacctgcaccgtgagcgccggcagcatcagcagcaccagcaccagctactactggggctggatcaggcagagccccggcaagggcctggagtggatcggcagcatctactacagcggcaggacctactacaaccccagcctgaagagcagggtgactatcagcgtggacaggcccaacaaccagttcagcctgaaggtgagcagcgtgaccgccgccgacagcgccgtgtactactgcgccaggcacaggagggtgctgctgtggatcggcgagctgctgaacaactacaacaggaacgtgtggggccagggcacaatggtgaccgtgagcagcggtggtggaggcagcgggggtggaggtagcggcggaggtggtagctgtagcgcttagtaagcggccgcgtgctagc

SEQ ID NO: 52 - DNA, 1113 bp, synthetic construct
  source 1..1113: mol_type = other DNA
atgggcagcagccatcatcatcatcatcacgctgctgctgccgaggtccagctgcaggcgtctgggggaggattggtgcaggctggggactctctgagactctcttgtgcagcctctggaggcaccttcagtcactatgccgtgggctggttccgccaggctccagggaaggagcgtgagttcgtagcagctattagctggagtggtcgtagcacaagctttgcaaactccgtgaagggccgattcaccatctccagagacagcgccaagaatacggcctatctacaaatgaacaacctgaaacctgaggacacggccgtttattgttgtgcaccagcccgatttggtactggatcagcggcgcgggatgagtatgacgactgcggccaggggacccaggtcaccgtgtctagcgcggccgctagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 53 - DNA, 1107 bp, synthetic construct
  source 1..1107: mol_type = other DNA
atgggcagcagccatcatcatcatcatcacgctgctgctgctcaggtgcagctcgtggagtccgggggaggattggtgcaggctgggggctctctgaaactctcctgtgcagcctctggattcaccttcgctcactatgctatggtctggttccgccaggctccagggaaggagcgtgagtttgtagcaggcattagctggagtggtgctagcacatactatgcaagctccgtgaagggccgattcaccatctccagagacaacgccaagaacacggtgtatctgcaaatgaacagcctgaaacctgaggacacggccgtttattacgttgcagcggctcgttttggggttggggtcgacgatgactattcctactggggccaggggacccaggtcaccgtgtctagcgcggccgctagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 54 - protein, 21 aa, synthetic construct
AAARSDKTHTCPPCPAPELLG

SEQ ID NO: 55 - protein, 20 aa, synthetic construct
GGGGSGGGGSGGGGSGGGGS

SEQ ID NO: 56 - protein, 18 aa, synthetic construct
GGGGSGGGGSGGGGSCSA

SEQ ID NO: 57 - protein, 502 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 58 - protein, 14 aa, synthetic construct
MGSSHHHHHHAAAA

SEQ ID NO: 59 - protein, 263 aa, synthetic construct
EVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSS

SEQ ID NO: 60 - protein, 177 aa, synthetic construct
MKYLLPTAAAGLLLLAAQPAMAQVQLVESGGGLVQAGGSLKLSCAASGFTFAHYAMVWFRQAPGKEREFVAGISWSGASTYYASSVKGRFTISRDNAKNTVYLQMNSLKPEDTAVYYVAAARFGVGVDDDYSYWGQGTQVTVSSAHHSEDPSAAAEQKLISEEDLNGAAHHHHHHGS

SEQ ID NO: 61 - protein, 162 aa, synthetic construct
MKYLLPTAAAGLLLLAAQPAMAEVQLQASGGGLVQAGDSLRLSCAASGGTFSHYAVGWFRQAPGKEREFVAAISWSGRSTSMKYLLPTAAAGLLLLAAQPAMAEVQLQASGGGLVQAGDSLRLSCAASGGTFSHYAVGWFRQAPGKEREFVAAISWSGRSTS

SEQ ID NO: 62 - protein, 42 aa, synthetic construct
MKYLLPTAAAGLLLLAAQPAMGSSHHHHHHSSGLVPRGSAAA

SEQ ID NO: 63 - protein, 150 aa, synthetic construct
MAEVQLQASGGGLVQAGGSLRLSCAASGGTFSSYAIGWFRQTPGKEREFVAAISWSGVSTDYAGSVKGRFTISRDNAKNTVYLQMNSLKPEDTAVYYCAAARFGTSSPDDEYHYWGHGTQVTVSSAAAEQKLISEEDLNGAAHHHHHHGS

SEQ ID NO: 64 - protein, 149 aa, synthetic construct
MADVQLVESGGGLVQTGGSLRVSCAPSGRTVGTYTMGWFRQAPGENRDFVASISGAVGTTYYADSVQGRFTISRDNAKNTVYLQMNSLKPEDTAVYYCAAGSFPLTRTNYVQFWGQGTQVTVSSAAAEQKLISEEDLNGAAHHHHHHGS

SEQ ID NO: 65 - protein, 157 aa, synthetic construct
MAQVQLVESGGGLVQAGGSLKLSCAASGFTFAHYAMVWFRQAPGKEREFVAGISWSGASTYYASSVKGRFTISRDNAKNTVYLQMNSLKPEDTAVYYVAAARFGVGVDDDYSYWGQGTQVTVSSAHHSEDPSAAAEQKLISEEDLNGAAHHHHHHGS

SEQ ID NO: 66 - protein, 150 aa, synthetic construct
MAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSAAEQKLISEEDLNGAAHHHHHHGS

SEQ ID NO: 67 - protein, 160 aa, synthetic construct
MAQLQLVESGGGLVQPGGSLRLSCAASGFTLDYYTIGWFRQAPGKEREGVSCIRGSGGSTNYADSVKGRFTISRDNAKNTVYLQMNSLKPEDTAVYYCAADFLSTCSLAGYRYEEVWGQGTLVTVSSAHHSEDPSAAAEQKLISEEDLNGAAHHHHHHGS

SEQ ID NO: 68 - protein, 157 aa, synthetic construct
MAEVQLVESGGGLVQAGGSLRLSCAAPGITLSRYGMYGMGWFRQAPGKEREFVAAITWSGGQTYYQDSVKGRFTISRDNAKKLTFLQMNSLKPEDTAVYYCAADGDRFYPEPVVDDNAYKFWGQGTQVTVSSAAAEQKLISEEDLNGAAHHHHHHGS

SEQ ID NO: 69 - protein, 153 aa, synthetic construct
MADVQLVESGGGLVQAGGSLRLSCAASGRTFSSYSMGWFRQAPGKEREFVAAINWSGGTTYYADSVKGRFTISRDNAKNTVYLQMNSLKPEDTAVYYCAAGGTTAQGMSVMTPRLGSWGQGTQVTVSSAAAEQKLISEEDLNGAAHHHHHHGS

SEQ ID NO: 70 - protein, 268 aa, synthetic construct
MAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSEVQLVESGGGVVQPGGSLKLSCVASGTDFSINFIRWYRQAPGKQREFVAGFTATGNTNYADSMKGRFTISRDNTKNAVYLQIDSLKPEDTAVYYCYMLDKWGQGTHVTVSSGGGGSHHHHHHGSCSA

SEQ ID NO: 71 - protein, 268 aa, synthetic construct
MAEVQLQASGGGLVQAGDSLRLSCAASGGTFSHYAVGWFRQAPGKEREFVAAISWSGRSTSFANSVKGRFTISRDSAKNTAYLQMNNLKPEDTAVYCCAPARFGTGSAARDEYDDCGQGTQVTVSSGGGGSGGGGSGGGGSEVQLVESGGGVVQPGGSLRLSCAASGEIFSINFMRWYRQAPGKQREWVAGFTRDGSTNYPDSAKGRFTISRDNAKNTVYLQIDSLKPEDTAVYYCYMLDTWGQGTQVTVSSGGGGSHHHHHHGSCSA

SEQ ID NO: 72 - protein, 24 aa, synthetic construct
EVQLQASGGGLVQAGGSLRLSCAA

SEQ ID NO: 73 - protein, 24 aa, synthetic construct
DVQLVESGGGLVQTGGSLRVSCAP

SEQ ID NO: 74 - protein, 24 aa, synthetic construct
QVQLVESGGGLVQAGGSLKLSCAA

SEQ ID NO: 75 - protein, 24 aa, synthetic construct
EVQLQASGGGLVQAGDSLRLSCAA

SEQ ID NO: 76 - protein, 24 aa, synthetic construct
QLQLVESGGGLVQPGGSLRLSCAA

SEQ ID NO: 77 - protein, 24 aa, synthetic construct
EVQLVESGGGLVQAGGSLRLSCAA

SEQ ID NO: 78 - protein, 24 aa, synthetic construct
DVQLVESGGGLVQAGGSLRLSCAA

SEQ ID NO: 79 - protein, 14 aa, synthetic construct
WFRQTPGKEREFVA

SEQ ID NO: 80 - protein, 14 aa, synthetic construct
WFRQAPGENRDFVA

SEQ ID NO: 81 - protein, 14 aa, synthetic construct
WFRQAPGKEREFVA

SEQ ID NO: 82 - protein, 14 aa, synthetic construct
WFRQAPGKEREFVA

SEQ ID NO: 83 - protein, 14 aa, synthetic construct
WFRQAPGKEREGVS

SEQ ID NO: 84 - protein, 14 aa, synthetic construct
WFRQAPGKEREFVA

SEQ ID NO: 85 - protein, 14 aa, synthetic construct
WFRQAPGKEREFVA

SEQ ID NO: 86 - protein, 33 aa, synthetic construct
VKGRFTISRDNAKNTVYLQMNSLKPEDTAVYYC

SEQ ID NO: 87 - protein, 34 aa, synthetic construct
SVQGRFTISRDNAKNTVYLQMNSLKPEDTAVYYC

SEQ ID NO: 88 - protein, 34 aa, synthetic construct
SVKGRFTISRDNAKNTVYLQMNSLKPEDTAVYYV

SEQ ID NO: 89 - protein, 34 aa, synthetic construct
SVKGRFTISRDSAKNTAYLQMNNLKPEDTAVYCC

SEQ ID NO: 90 - protein, 34 aa, synthetic construct
SVKGRFTISRDNAKNTVYLQMNSLKPEDTAVYYC

SEQ ID NO: 91 - protein, 33 aa, synthetic construct
SVKGRFTISRDNAKKLTFLQMNSLKPEDTAVYY

SEQ ID NO: 92 - protein, 34 aa, synthetic construct; identical to SEQ ID NO: 90

SEQ ID NO: 93 - protein, 11 aa, synthetic construct
WGHGTQVTVSS

SEQ ID NO: 94 - protein, 11 aa, synthetic construct
WGQGTQVTVSS

SEQ ID NO: 95 - protein, 11 aa, synthetic construct
WGQGTQVTVSS

SEQ ID NO: 96 - protein, 11 aa, synthetic construct
CGQGTQVTVSS

SEQ ID NO: 97 - protein, 11 aa, synthetic construct
WGQGTLVTVSS

SEQ ID NO: 98 - protein, 11 aa, synthetic construct
WGQGTQVTVSS

SEQ ID NO: 99 - protein, 11 aa, synthetic construct
WGQGTQVTVSS